(19) United States
(12) Patent Application Publication
(10) Pub. No.: US 2023/0313223 A1
(43) Pub. Date: Oct. 5, 2023

(54) METHODS FOR MODULATING LEVEL OF EXPRESSION FROM GENE THERAPY EXPRESSION CASSETTE

(71) Applicant: Cornell University, Ithaca, NY (US)

(72) Inventors: Ronald G. CRYSTAL, New York, NY (US); Katie M. STILES, Bronx, NY (US)

(21) Appl. No.: 17/769,188

(22) PCT Filed: Oct. 15, 2020
(86) PCT No.: PCT/US2020/055832

§ 371 Date: Apr. 14, 2022

Related U.S. Application Data
(60) Provisional application No. 62/915,342, filed on Oct. 15, 2019.

(51) Int. Cl. C12N 15/86; A61K 48/00

(52) U.S. Cl. CPC C12N 15/86; A61K 48/0008; C12N 2750/14143

(57) ABSTRACT

Compositions having nucleic acid sequences for regulating expression from gene therapy vectors, and methods of using the compositions, are provided.

### CROSS-REFERENCE TO RELATED APPLICATIONS

This application claims the benefit of the filing date of U.S. application No. 62/915,342 filed on Oct. 15, 2019, the disclosure of which is incorporated by reference herein.

### BACKGROUND

A current challenge in gene therapy is controlling the level of expression from the transgene expression cassette once the vector has been delivered. In the current state, once a gene transfer vector has been administered to the recipient, the expression from the transgene cannot be modulated, shut off, or turned on. Controlling the transgene expression from administered vectors is important for safety and better efficacy. Expression of some types of transgenes may only need to be expressed intermittently. Other transgenes may need to be shut off because of adverse reactions in individual patients.

### SUMMARY

The present disclosure provides for gene therapy expression cassettes that contain ‘switches’ that allow expression to be turned on or off by the delivery of exogenous small non-coding RNAs. The gene therapy vector transgene expression constructs incorporate specific targeting, e.g., highly specific targeting, sequences for in one embodiment, small non-coding RNAs. In one embodiment, targeting sequences in the 3′ UTR of the cassette act as a switch to turn gene expression off, e.g., intermittently, in the presence of exogenous small interfering RNA (siRNA) directed towards the highly specific sequence. In one embodiment, targeting sequences in the 3′ UTR of the cassette in a vector such as a viral vector act as a switch to turn gene expression off, e.g., permanently, in the presence of exogenous micro RNA (miRNA) directed towards the highly specific sequence. In one embodiment, targeting sequences incorporated into hairpin(s) in the 5′ UTR act as a switch to turn on gene expression in the presence of exogenous small RNAs directed towards the specific sequence.

In one embodiment, small non-coding RNAs including siRNA sequences. e.g., shRNA sequences, can reduce gene expression by binding to a specific complementary, e.g., 21 to 25 nucleotides, sequence in a mRNA transcript thus directing the mRNA for destruction and reducing or eliminating protein output for the corresponding gene. The incorporation of highly specific siRNA targeting sequences into the 3′ untranslated region (UTR) of the transgene expression cassette allows transgene expression to be shut off with the delivery of exogenous siRNAs specific for the target sequence. Transgene expression would remain off as long as these siRNAs are present and expression could be resumed by ceasing delivery of the siRNA. Because the highly specific siRNA targeting sequence is encoded in the 3′ UTR of transgene cassette, this strategy can be used universally with any promoter and transgene combination.

In one embodiment, small non-coding RNAs including miRNA sequences can reduce gene expression by binding to a specific complementary nucleotide sequence in a mRNA transcript thus directing the mRNA for destruction and reducing or eliminating protein output for the corresponding gene. The incorporation of highly specific miRNA targeting sequences into the 3′ untranslated region (UTR) of the transgene expression cassette allows transgene expression to be shut off with the delivery of exogenous miRNAs specific for the target sequence. e.g., encoded by a viral vector. Transgene expression would remain off as long as these miRNAs are present. e.g., if expressed from a constitutive promoter, transgene expression would silenced permanently. Because the highly specific miRNA targeting sequence is encoded in the 3′ UTR of transgene cassette, this strategy can be used universally with any promoter and transgene combination.

In one embodiment, small non-coding RNAs can also act to increase gene expression by acting as triggers to activate protein translation from the targeted mRNA using, for example, a toehold-like switch. Hairpin(s) in the mRNA 5′ UTR upstream of the AUG start codon include the small RNA target site, a linker, and a sequence complementary to the small RNA target site which act to block ribosome scanning and protein translation, e.g., by forming a hairpin, effectively eliminating transgene expression. Delivery of small RNAs that bind to the target site opens the hairpin allowing for ribosome scanning and protein translation. Transgene expression would remain on only in the presence of the exogenous small triggering RNAs. The incorporation of the hairpins into the 5′ UTR of the transgene cassette allows this strategy to be applied to any promoter and transgene combination used for gene therapy.

In one embodiment, the disclosure provides a gene therapy vector having one or more regions for control of expression of a linked therapeutic or prophylactic gene, where the regions for control have one or more sequences that flank the gene, wherein sequences in the regions can interact with short RNA sequences that allow for translation of the corresponding transcribed RNA, inhibition of translation of corresponding transcribed RNA and/or degradation of the corresponding transcribed RNA, or enhanced expression of the transcribed RNA. In one embodiment, the disclosure provides a method of using the vector and short RNA sequences.

In one embodiment, a gene therapy vector is provided comprising a promoter operably linked to a nucleic acid sequence comprising an open reading frame encoding a prophylactic or therapeutic gene product and a 3′ untranslated region (3′ UTR), wherein the vector further comprises a first transcriptional regulatory region 3′ to the open reading frame that, when present in transcribed RNA, is capable of i) interacting with a short interfering RNA (siRNA) sequence which interaction inhibits translation of the transcribed RNA or 2) enhancing degradation of the transcribed RNA, thereby inhibiting the expression of the gene product, and/or wherein the vector further comprises a second transcriptional regulatory region 5′ to the open reading frame that, when present in transcribed RNA, is capable of interacting with a short activating RNA (saRNA; trigger RNA) sequence which interaction allows for translation of the transcribed RNA, thereby allowing for expression of the gene product. In one embodiment, the vector has the first transcriptional regulatory region but not the second transcriptional regulatory region. In one embodiment, the vector has the second transcriptional regulatory region but not the first transcriptional regulatory region. In one embodiment, the vector has the first transcriptional regulatory region and the second transcriptional regulatory region. In one embodiment, the vector is a viral vector. In one embodiment, the viral vector is an AAV, adenovirus, lentivirus, herpesvirus or retrovirus vector. In one embodiment, the transcribed RNA having the second transcriptional regulatory region forms at least one hairpin structure. In one embodiment, the interaction of the saRNA with the transcribed RNA having the second transcriptional regulatory region exposes the ribosome binding site. In one embodiment, the transcribed RNA having the second transcriptional regulatory region forms one to three or two to five different hairpins. In one embodiment, the transcribed RNA having the second transcriptional regulatory region forms at least one hairpin that overlaps the ribosome binding site and/or the first AUG in the transcribed RNA. In one embodiment, the second transcriptional regulatory region comprises a toe hold sequence, e.g., which sequence includes a recognition site for an RNA (an activating RNA; saRNA), and a site for ribosome binding site (RBS) and/or that includes the first AUG in an open reading frame, that in transcribed RNA form a hairpin and one or more loops (single stranded regions) that include the RBS and/or first AUG in the absence of the saRNA, thus inhibiting translation, but which sequence when present in transcribed RNA and in the presence of the saRNA, allows for translation. In one embodiment, the first transcriptional regulatory region comprises more than one nucleotide sequence that when transcribed into RNA can bind more than one distinct siRNA. In one embodiment, the first transcriptional regulatory region comprises a nucleotide sequence that when transcribed into RNA can bind a plurality of siRNAs. In one embodiment, the nucleotide sequence has a plurality of siRNA binding sites for the same siRNA. In one embodiment, the open reading frame encodes a therapeutic RNA, a therapeutic antibody, an anti-cancer gene product, a complement factor, an interleukin, a cytokine, or a hormone. In one embodiment, the gene product comprises an anti-EGFR antibody, anti-VEGF antibody, anti-VEGFR antibody, alpha 1-antitrypsin, catalase, superoxide dismutase, factor 9, IL-2R, adenosine deaminase (ADA), WAS, beta-globin, ABCD1, anti-CD19 antibody, or FK506 binding protein. In one embodiment, the first transcriptional regulatory region comprises a sequence that binds to a siRNA having at least 80% nucleotide sequence identity with one of SEQ ID Nos. 1 or 2.

In one embodiment, a system is provided comprising: gene therapy vector comprising a promoter operably linked to a nucleic acid sequence comprising an open reading frame encoding a prophylactic or therapeutic gene product and a 3′ untranslated region (3′ UTR), wherein the vector further comprises a first transcriptional regulatory region 3′ to the nucleic acid sequence that, when present in transcribed RNA, is capable of interacting with a short interfering RNA (siRNA) sequence which interaction inhibits translation of the transcribed RNA or 2) enhancing degradation of the transcribed RNA, thereby inhibiting the expression of the gene product, and siRNA. In one embodiment, the siRNA is expressed from a vector. In one embodiment, the siRNA is expressed from a viral vector. In one embodiment, the siRNA comprises isolated siRNA. In one embodiment, the first transcriptional regulatory region comprises a sequence that binds to a siRNA having at least 80% nucleotide sequence identity with one of SEQ ID Nos. 1 or 2.

In one embodiment, a system is provided comprising: a gene therapy vector comprising a promoter operably linked to a nucleic acid sequence comprising an open reading frame encoding a prophylactic or therapeutic gene product and a 3′ untranslated region (3′ UTR), wherein the vector further comprises a second transcriptional regulatory region 5′ to the nucleic acid sequence that, when present in transcribed RNA, is capable of interacting with a short activating RNA (saRNA) sequence which interaction allows for translation of the transcribed RNA, thereby allowing for expression of the gene product; and a vector comprising sequences corresponding to the saRNA. In one embodiment, the second transcriptional regulatory region comprises a toe hold sequence.

In one embodiment, a method to control expression, e.g., regulate or rmodulate expression, by increasing or decreasing expression, of a gene therapy vector in a mammal is provided. The method includes providing a mammal having the gene therapy vector; and administering to the mammal an amount of a composition comprising nucleic acid comprising sequences for the siRNA and/or the saRNA effective to alter expression of the gene product encoded by the vector. In one embodiment, the open reading frame encodes a protein. In one embodiment, the open reading frame encodes a therapeutic RNA. In one embodiment, the gene product is a therapeutic antibody, a hormone, a cytokine, an interleukin, or a ribozyme. In one embodiment, the composition comprises siRNA optionally having one or more nucleotide analogs. In one embodiment, the composition comprises a DNA vector having sequences corresponding to the siRNA sequences or the saRNA sequences. In one embodiment, the composition comprises a plurality of distinct siRNAs. In one embodiment, the composition comprises isolated saRNA. In one embodiment, the composition comprises a plurality of distinct saRNAs. In one embodiment, the composition comprises a nucleic acid vector comprising the nucleic acid comprising sequences the siRNA or saRNA. In one embodiment, the composition comprises liposomes comprising the nucleic acid. In one embodiment, the composition comprises nanoparticles comprising the nucleic acid. In one embodiment, the composition comprises protein complexes comprising the nucleic acid. In one embodiment, the composition is systemically administered. In one embodiment, the composition is orally administered. In one embodiment, the composition is intravenously administered. In one embodiment, the composition is locally administered. In one embodiment, the composition is injected. In one embodiment, the composition is a sustained release composition. In one embodiment, the method further comprises administering the gene therapy vector to the mammal. In one embodiment, liposomes comprise the gene therapy vector. In one embodiment, nanoparticles comprise the gene therapy vector. In one embodiment, protein complexes comprising the gene therapy vector. In one embodiment, virus comprises the gene therapy vector. In one embodiment, the vector is intravenously administered. In one embodiment, the vector is locally administered. In one embodiment, the mammal is a human.

Also provided is a vector having homology arms flanking a second transcriptional regulatory region that when present in transcribed RNA is capable of interacting with a short activating RNA (saRNA) sequence, wherein the homology arms have sequences that correspond to gene therapy vector sequences that flank a site for insertion of the second transcriptional regulatory region. In one embodiment, the site for insertion is 3′ to the promoter and 5′ to an open reading frame for a prophylactic or a therapeutic gene product.

Further provided is a vector having homology arms flanking a first transcriptional regulatory region that when present in transcribed RNA is capable of interacting with a short interfering RNA (siRNA) sequence, wherein the homology arms have sequences that correspond to gene therapy vector sequences that flank a site for insertion of the first transcriptional regulatory region. In one embodiment, the site for insertion is 3′ to an open reading frame for a prophylactic or a therapeutic gene product and 5′ to the 3′ end of a 3′UTR.

### BRIEF DESCRIPTION OF THE FIGURES

FIG. 1. Exemplary universal AAV vector inhibition using inhibitory siRNA.

FIG. 2. RNA triggered hairpin switch to turn on expression. A stable hairpin the overlapping ribosome binding site and AUG start codon blocks protein expression. In one embodiment, multiple hairpins may be employed to avoid leaky expression without trigger RNAs. Binding of a trigger RNA (e.g., about 23 to 30 nt in length) releases the hairpin, which in turn allows ribosomes to bind, resulting in expression.

FIG. 3. Exemplary universal AAV vector for intermittent inhibition of expression using inhibitory siRNA.

FIG. 4. Exemplary universal AAV vector for permanent inhibition of expression using a second vector, e.g., an AAV vector.

FIG. 5. Exemplary vector for regulatable Epo expression.

FIGS. 6A-6B. A) Measurement of EPO levels. B) Dose dependent inhibition of expression.

FIG. 7. Mouse study.

FIG. 8. Exposure of allergic individuals to the allergen to which they are sensitized results in allergen-specific IgE-allergen immune complexes that bind to mast cells, mediating the anaphylactic reaction. Gene therapy uses, in one embodiment, an AAV serotype rh.10 gene transfer vector coding for heavy and light chains of omalizumab (Pagovich et al., 2016). Following IV administration, the vector modifies hepatocytes to persistently express omalizumab, providing persistent therapy to block anti-IgE-allergen activation of mast cells.

FIGS. 9A-9D. Treatment of established peanut antigen-induced systemic anaphylaxis with vector07. A) Persistent expression following single administration of vector07. B-D) Efficacy data in a humanized peanut-allergic mouse model (Pagovich et al., 2016).

FIG. 10. Gene transfer vectors. Vector07A (AAVrh.10anti-IgE-T) is the therapeutic vector expressing anti-IgE. Vector07 and Vector07A use the CAG highly active constitutive promoter driving a secretion signal followed by the anti-IgE omalizumab heaving and light chain separated by a furin 2A site; when expressed, the furin 2A site is cleaved and tshe heavy and light chain combine to generate functional anti-IgE which is secreted (Pagovich et al., 2016). Vector07A is identical to Vector07 except it has 3′ to the anti-IgE heavy and light chain, 5, 21 bp tandem target sequences to the cognate miRNA expressed by Vector09A and Vector09B. Vector09A (AdC7miRNA-E) is a serotype E1⁻E3⁻ AdC7 vector coding for 8 tandem miRNA sequences cognate to the Vector07A target sequence. Vector09B (AAV5miRNA-E) in a serotype 5 AAV vector coding for the same tandem miRNA sequences as in Vector09A. Vector09A and Vector09B also express reporter genes (mCherry, EGFP, respectively) so transfected liver cells can be identified.

FIG. 11. Function of the unique miRNA targeting sequence. Plasmid coding for the 21 bp miRNA targeting sequence (33 ng/well) was transfected is 293T cells±cognate siRNA (5 pmol). After 24 hours, the CAG-driven reporter expression was quantified by Western.

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots. Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences. e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures.” “packaging cell line” and other such terms denote higher eukaryotic cells, such as mammalian cells including human cells, useful in the present invention. e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19. P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette.” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature. e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence. e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G. U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

### Vectors and Methods

In conventional use, once a gene transfer vector has been administered to a recipient, the expression from the transgene cannot be modulated, shut off or turned on. Control of previously administered vectors is important for safety if there is too much expression or efficacy in having the vector turned on when needed.

Expression of some types of transgenes (e.g., therapeutic antibodies, hormones, anti-tumor therapies and the like) may not be needed at all times. Small non-coding RNAs can be used to decrease and increase expression of genes. Extensive technology has been developed regarding the in vivo administration of non-coding RNAs to regulate gene expression, and small non-coding RNAs have been incorporated into gene transfer vectors to actively suppress genes. The present disclosure provides for the use of small non-coding RNAs delivered independently after vector-mediated gene transfer to control (on or off) the expression of the gene that has been transferred. Incorporation in the vector expression cassette of targeting sites for small interfering RNAs (siRNA) or small activating RNAs (saRNA) would allow for control of transgene expression of a previously administered vector/expression cassette as needed by administering of exogenous siRNA or sa RNA.

### Exemplary Gene Therapy Vectors

The disclosure provides a gene therapy vector comprising a nucleic acid sequence having an open reading frame that encodes a therapeutic or a prophylactic gene product and a nucleic acid sequence having a first transcriptional regulatory region 3′ to the open reading frame that, when present in transcribed RNA, is capable of i) interacting with a short interfering RNA (siRNA) sequence which interaction inhibits translation of the transcribed RNA or 2) enhancing degradation of the transcribed RNA, thereby inhibiting the expression of the gene product, and/or the vector comprises a second transcriptional regulatory region 5′ to the open reading frame that, when present in transcribed RNA, is capable of interacting with a short activating RNA (saRNA) sequence which interaction allows for translation of the transcribed RNA, thereby allowing for expression of the gene product. Also provided are compositions comprising siRNA or a vector for expression siRNA, or comprising saRNA or a vector for expression of saRNA.

Various aspects of the gene therapy vector, transcriptional regulatory regions, siRNA and/or saRNA, and methods are discussed below. Although each parameter is discussed separately, the gene therapy vector, the transcriptional regulatory region(s), siRNA, and/or saRNA, and methods comprise combinations of the parameters set forth below to evoke control of a therapeutic or a prophylactic gene product. Accordingly, any combination of parameters can be used according to the gene therapy vector, transcriptional regulatory region(s), siRNA and/or saRNA, and the methods.

A “gene therapy vector” is thus any molecule or composition that has the ability to carry a heterologous nucleic acid sequence into a suitable host cell where synthesis of the encoded protein takes place. Typically, a gene therapy vector is a nucleic acid molecule that has been engineered, using recombinant DNA techniques that are known in the art, to incorporate the heterologous nucleic acid sequence. Desirably, the gene therapy vector is comprised of DNA. Examples of suitable DNA-based gene therapy vectors include plasmids and viral vectors. However, gene therapy vectors that are not based on nucleic acids, such as liposomes or nanoparticles having the gene therapy vectors and one or more of the transcriptional regulatory regions, may be employed. The gene therapy vector can be based on a single type of nucleic acid (e.g., a plasmid) or non-nucleic acid molecule (e.g., a lipid or a polymer). The gene therapy vector can be integrated into the host cell genome, or can be present in the host cell in the form of an episome.

Gene delivery vectors within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch.

In one embodiment, the gene therapy vector or the vector for siRNA or saRNA expression is a viral vector. Suitable viral vectors include, for example, retroviral vectors, lentivirus vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons. New York, N.Y. (1994).

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells. Adenoviral vectors have been shown to result in transient expression of therapeutic genes in vivo, peaking at 7 days and lasting approximately 4 weeks. In addition, adenoviral vectors can be produced at very high titers, allowing efficient gene therapy with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh10, including chimeric viruses where the AAV genome is from a different source than the capsid.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Exemplary AAV Vectors

In an embodiment, the disclosure provides an adeno-associated virus (AAV) vector which comprises, consists essentially of, or consists of a nucleic acid sequence comprising a nucleic acid sequence having an open reading frame that encodes a therapeutic or a prophylactic gene product and a nucleic acid sequence having a first transcriptional regulatory region 3′ to the open reading frame that, when present in transcribed RNA, is capable of i) interacting with a short interfering RNA (siRNA) sequence which interaction inhibits translation of the transcribed RNA or 2) enhancing degradation of the transcribed RNA, thereby inhibiting the expression of the gene product, and/or the vector comprises a second transcriptional regulatory region 5′ to the open reading frame that, when present in transcribed RNA, is capable of interacting with a short activating RNA (saRNA) sequence which interaction allows for translation of the transcribed RNA, thereby allowing for expression of the gene product. When the AAV vector consists essentially of a nucleic acid sequence encoding the gene product and one or both transcriptional regulatory regions, additional components can be included that do not materially affect the AAV vector (e.g., genetic elements such as poly(A) sequences or restriction enzyme sites that facilitate manipulation of the vector in vitro). When the AAV vector consists of a nucleic acid sequence which encodes the gene product and one or both of the transcriptional regulatory regions, the AAV vector does not comprise any additional components (i.e., components that are not endogenous to AAV and are not required to effect expression of the nucleic acid sequence).

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61:447 (1990)). These proteins also regulate transcription from endogenous AAV promoters and promoters within helper viruses (see, e.g., Pereira et al., J. Virol., 71:1079 (1997)). The other Rep proteins modify the function of Rep78 and Rep68. The cap genes encode the capsid proteins VP1, VP2, and VP3. The cap genes are transcribed from the p40 promoter.

The AAV vector may be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-5 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, Hum. Gene Ther., 16:541 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71:6823 (1997); Srivastava et al., J. Virol., 4:555 (1983); Chiorini et al., J. Virol., 73:1309 (1999); Rutledge et al., J. Virol., 72:309 (1998); and Wu et al., J. Virol., 74:8635 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 71(2):939 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (e.g., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 11(1):1 (2006); Gao et al., J. Virol., 7:6381 (2004); Gao et al., Proc. Natl. Acad. Sci. USA, 99:11854 (2002); De et al., Mol. Ther., 13:67 (2006); and Gao et al., Mol. Ther., 13:77 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In one embodiment, the AAV vector is generated using an AAV that infects a non-human primate pseudotyped with an AAV that infects humans. Examples of such pseudotyped AAV vectors are disclosed in, e.g., Cearley et al., Molecular Therapy, 13:528 (2006). In one embodiment, an AAV vector can be generated which comprises a capsid protein from an AAV that infects rhesus macaques pseudotyped with AAV2 inverted terminal repeats (ITRs). In a particular embodiment, the AAV vector comprises a capsid protein from AAV10 (also referred to as “AAVrh.10”), which infects rhesus macaques pseudotyped with AAV2 ITRs (see, e.g., Watanabe et al., Gene Ther., 17(8):1042 (2010); and Mao et al., Hum. Gene Therapy, 22:1525 (2011)).

In addition to the nucleic acid sequence encoding gene product and one or more transcriptional regulatory regions, the AAV vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185. Academic Press, San Diego, CA. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 91:3346 (1996)), the T-REXTM system (Invitrogen, Carlsbad, CA), LACSWITCH™ System (Stratagene, San Diego, CA), and the Cre-ERT tamoxifen inducible recombinase system (Indra et al., Nuc. Acid. Res., 27:4324 (1999); Nuc. Acid. Res., 28:e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence encoding EPO or an antibody, is operably linked to a CMV enhancer/chicken beta-actin promoter (also referred to as a “CAG promoter”) (see, e.g., Niwa et al., Gene, 108:193 (1991); Daly et al., Proc. Natl. Acad. Sci. U.S.A., 96:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested and the vector is released from the cells by five freeze/thaw cycles. Subsequent centrifugation and benzonase treatment removes cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1×phosphate buffered saline. The viral titers may be measured by TaqMan® real-time PCR and the viral purity may be assessed by SDS-PAGE.

### Pharmaceutical Compositions and Delivery

The disclosure provides a composition comprising, consisting essentially of, or consisting of the above-described gene therapy vector and a pharmaceutically acceptable (e.g., physiologically acceptable) carrier, and/or comprising siRNA, saRNA, or vectors for expression of siRNA and/or saRNA. When the composition consists essentially of, for example, the gene therapy vector and a pharmaceutically acceptable carrier, additional components can be included that do not materially affect the composition (e.g., adjuvants, buffers, stabilizers, anti-inflammatory agents, solubilizers, preservatives, etc.). When the composition consists of, for example, the gene therapy vector and the pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene therapy vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 2/st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene therapy vector, siRNA or saRNA is administered in a composition formulated to protect the gene therapy vector, siRNA or saRNA from damage prior to or after administration. For example, the composition can be formulated to reduce loss of the gene therapy vector on devices used to prepare, store, or administer the gene therapy vector, such as glassware, syringes, or needles. The composition can be formulated to decrease the light sensitivity and/or temperature sensitivity of the gene therapy vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene therapy vector, facilitate administration, and increase the efficiency of the method. Formulations for gene therapy vector-containing compositions are further described in, for example. Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the gene therapy vector can be present in a composition with other therapeutic or biologically-active agents. For example, factors that control inflammation, such as ibuprofen or steroids, can be part of the composition to reduce swelling and inflammation associated with in vivo administration of the gene therapy vector. Immune system stimulators or adjuvants, e.g., interleukins, lipopolysaccharide, and double-stranded RNA, can be administered to enhance or modify an immune response. Antibiotics, i.e., microbicides and fungicides, can be present to treat existing infection and/or reduce the risk of future infection, such as infection associated with gene therapy procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see. e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene therapy vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene therapy vectors, or siRNA and/or saRNA, or vectors for expression thereof, may be intracerebral (including but not limited to intraparenchymal, intraventricular, or intracisternal), intrathecal (including but not limited to lumbar or cisterna magna), or systemic, including but not limited to intravenous, or any combination thereof, using devices known in the art. Delivery may also be via surgical implantation of an implanted device.

The dose of the gene therapy vector, siRNA or saRNA, or vectors for expression thereof, in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the gene therapy vector described herein. A “therapeutically effective amount” refers to an amount effective, at dosages and for periods of time necessary, to achieve a desired therapeutic result. The therapeutically effective amount may vary according to factors such as the extent of pathology, age, sex, and weight of the individual, and the ability of the gene therapy vector to elicit a desired response in the individual. The dose of gene therapy vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene therapy vector dose range to treat a patient having a particular disease or disorder, based on these and other factors that are well known in the art. The therapeutically effective amount may be between 1×10¹⁰ genome copies to 1×10¹³ genome copies. The therapeutically effective amount may be between 1×10¹¹ genome copies to 1×10¹⁴ genome copies. The therapeutically effective amount may be between 1×10¹² genome copies to 1×10¹⁵ genome copies. The therapeutically effective amount may be between 0.5 to 2×10¹³ genome copies (gc) to 0.5 to 2×10¹⁶ gc, e.g., a total dose in humans, e.g., 1×10¹³ gc to 1×10¹⁴gc, 1×10¹⁴ gc to 1×10¹⁵ gc or 1×10¹⁵ gc to 1×10¹⁴gc. In one embodiment, a total dose in humans is from about 1×10⁸ gc/kg to about 2×10¹⁰gc/kg, e.g., 1.5×10⁹ gc/kg to about 1.5×10¹¹ gc/kg, 1.5×10¹⁰ gc/kg to about 1.5×10¹² gc/kg or 1.5×10¹² gc/kg to about 1.5×10¹³ gc/kg. Assuming a 70 kg human, the dose ranges may be from 1.4×10⁸ gc/kg to 1.4×10¹¹ gc/kg, 1.4×10⁹ gc/kg to 1.4×10¹² gc/kg, 1.4×10¹⁰ gc/kg to 1.4×10¹³ gc/kg, or 1.4×10¹¹ gc/kg to 1.4×10¹⁴ gc/kg.

In one embodiment, the composition comprising the gene therapy vector, siRNA or saRNA, or vectors for expression hereof, is administered once to the mammal. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

In one embodiment, subjects include human subjects suffering from or at risk for the medical diseases and disorders described herein. The subject is generally diagnosed with the condition by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians. Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of treatment, as described above. In addition, the subject can have the genotype or phenotype for any hair color, eye color, skin color or any combination thereof. The term subject includes a subject of any body height, body weight, or any organ or body part size or shape.

### Exemplary Nanoparticle Formulations

Biodegradable nanoparticles. e.g., comprising the gene therapy vector or isolated siRNA or saRNA nucleic acid, or a vector for expression of siRNA or saRNA, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA. PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See. e.g., Kumari et al., Colloids and Surfaces B; Biointerfaces 75 (2010) 1-18; and U.S. Pat. Nos., 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U,S, Published Application No, 2007/0081972; and International Application Publication Nos. WO 2012/115806; and WO 2012/054425; the contents of which are incorporated herein by reference in their entireties).

The biodegradable nanoparticles may be prepared by methods known in the art. (See. e.g., Nagavarma et al., Asian J. of Pharma. And Clin. Res., Vol 5, Suppl 3, 2012, pages 16-23: Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS). Nanoparticle Tracking Analysis (NTA), X-Ray Diffraction (XRD), Aerosol Time of Flight Mass Spectroscopy (ATFMS), and Aerosol Particle Mass Analyzer (APM).

The biodegradable nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC). DOPE, or DC-cholesterol; peptide based vectors including but not limited to Poly-L-lysine or protamine; or poly(β-amino ester), chitosan. PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethylencamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA. DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives. e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

### EXEMPLARY EMBODIMENTS

In one embodiment, a gene therapy vector is provided comprising promoter operably linked to a nucleic acid sequence comprising an open reading frame for a prophylactically or therapeutically useful gene product and a 3′ untranslated region (3′ UTR), wherein the vector further comprises a first transcriptional regulatory region 3′ to the nucleic acid sequence that, when present in transcribed RNA, is capable of interacting with a short interfering RNA (siRNA) sequence which interaction inhibits translation of the transcribed RNA or enhances degradation of the transcribed RNA, thereby inhibiting the expression of the gene product, and/or wherein the vector further comprises a second transcriptional regulatory region 5′ to the nucleic acid sequence that, when present in transcribed RNA, is capable of interacting with a short activating RNA (saRNA) sequence which interaction allows for translation of the transcribed RNA, thereby allowing for expression of the gene product. In one embodiment, the vector has the first but not the second transcriptional regulatory region. In one embodiment, the vector has the second but not the first transcriptional regulatory region. In one embodiment, the vector has the first and the second transcriptional regulatory region. In one embodiment, the vector is a viral vector. In one embodiment, the viral vector is an AAV, adenovirus, lentivirus, herpesvirus or retrovirus vector. In one embodiment, the second transcriptional regulatory region forms at least one hairpin structure. In one embodiment, the interaction of the saRNA with the second transcriptional regulatory region exposes the ribosome binding site. In one embodiment, the second transcriptional regulatory region forms one to three or two to five different hairpins. In one embodiment, the second transcriptional regulatory region forms at least one hairpin that overlaps the ribosome binding site and/or the first AUG in the transcribed RNA. In one embodiment, the second transcriptional regulatory region comprises a toe hold sequence. In one embodiment, the first transcriptional regulatory region comprises more than one nucleotide sequence that when transcribed into RNA can bind more than one distinct siRNA. In one embodiment, the first transcriptional regulatory region comprises a nucleotide sequence that when transcribed into RNA can bind a plurality of siRNAs. In one embodiment, the nucleotide sequence has a plurality of siRNA binding sites for the same siRNA. In one embodiment, the open reading frame encodes a therapeutic RNA, a therapeutic antibody, an anti-cancer gene product, a complement factor, an interleukin, a cytokine, or a hormone. In one embodiment, the gene product comprises an anti-EGFR antibody, anti-VEGF antibody, anti-VEGFR antibody, alpha 1-antitrypsin, catalase, superoxide dismutase, factor 9. IL-2R, adenosine deaminase (ADA), WAS, beta-globin, ABCD1, anti-CD19 antibody, or FK506 binding protein. In one embodiment, the vector includes at least one copy of SEQ ID Nos. 1 to 3, 7 to 9 or 17 to 20, or a nucleotide sequence with at least 80%, 85%, 90%, 92%, 94%, 95%, 98%, or 99% nucleotide sequence identity thereto. In one embodiment, the siRNA or miRNA sequence for inhibition or activation of RNA expressed from the vector includes at least one copy of SEQ ID Nos. 4 to 6 or 12 to 16, or a nucleotide sequence with at least 80%, 85%, 90%, 92%, 94%, 95%, 98%, or 99% nucleotide sequence identity thereto.

Further provided is a host cell comprising the vector. In one embodiment, the cell is a mammalian cell. In one embodiment, the cell is a human cell. In one embodiment, the host cell is in a host organism. In one embodiment, the host organism is a mammal. In one embodiment, the mammal is a non-human primate. In one embodiment, the mammal is a human.

Further provided is a method to control expression of a gene therapy vector in a mammal. The method includes providing a mammal having the gene therapy vector; and administering to the mammal an amount of a composition comprising nucleic acid comprising sequences for the siRNA and/or the saRNA effective to alter expression of the gene product encoded by the vector. In one embodiment, the open reading frame encodes a protein. In one embodiment, the open reading frame encodes a therapeutic RNA. In one embodiment, the gene product is a therapeutic antibody, a hormone, a cytokine, an interleukin, or a ribozyme. In one embodiment, the composition comprises RNA optionally having one or more nucleotide analogs. In one embodiment, the composition comprises a DNA vector having sequences corresponding to the siRNA sequences or the saRNA sequences. In one embodiment, the composition comprises a plurality of distinct siRNAs. In one embodiment, the composition comprises a plurality of distinct saRNAs.

Also provided is a method to control expression of a gene therapy vector in a mammal, comprising: introducing to a mammal the gene therapy vector; and administering to the mammal an amount of a composition comprising nucleic acid comprising sequences for the siRNA and/or the saRNA effective to alter expression of the gene product encoded by the vector. In one embodiment, the composition comprises liposomes comprising the nucleic acid. In one embodiment, the composition comprises nanoparticles comprising the nucleic acid. In one embodiment, the composition comprises protein complexes comprising the nucleic acid. In one embodiment, the composition is systemically administered. In one embodiment, the composition is orally administered. In one embodiment, the composition is intravenously administered. In one embodiment, the composition is locally administered. In one embodiment, the composition is injected. In one embodiment, the composition is a sustained release composition. In one embodiment, the vector is intravenously administered. In one embodiment, the vector is locally administered.

In one embodiment, a vector is provided having homology arms flanking a second transcriptional regulatory region that when present in transcribed RNA is capable of interacting with a short activating RNA (saRNA) sequence, wherein the homology arms have sequences that correspond to gene therapy vector sequences that flank a site for insertion of the second transcriptional regulatory region. In one embodiment, the site for insertion is 3′ to the promoter and 5′ to an open reading frame for a prophylactic or a therapeutic gene product.

In one embodiment, a vector is provided having homology arms flanking a first transcriptional regulatory region that when present in transcribed RNA is capable of interacting with a short interfering RNA (siRNA) sequence, wherein the homology arms have sequences that correspond to gene therapy vector sequences that flank a site for insertion of the first transcriptional regulatory region. In one embodiment, the site for insertion is 3′ to an open reading frame for a prophylactic or a therapeutic gene product and 5′ to the 3′ end of a 3′UTR.

The invention will be further described by the following non-limiting examples.

### Example 1

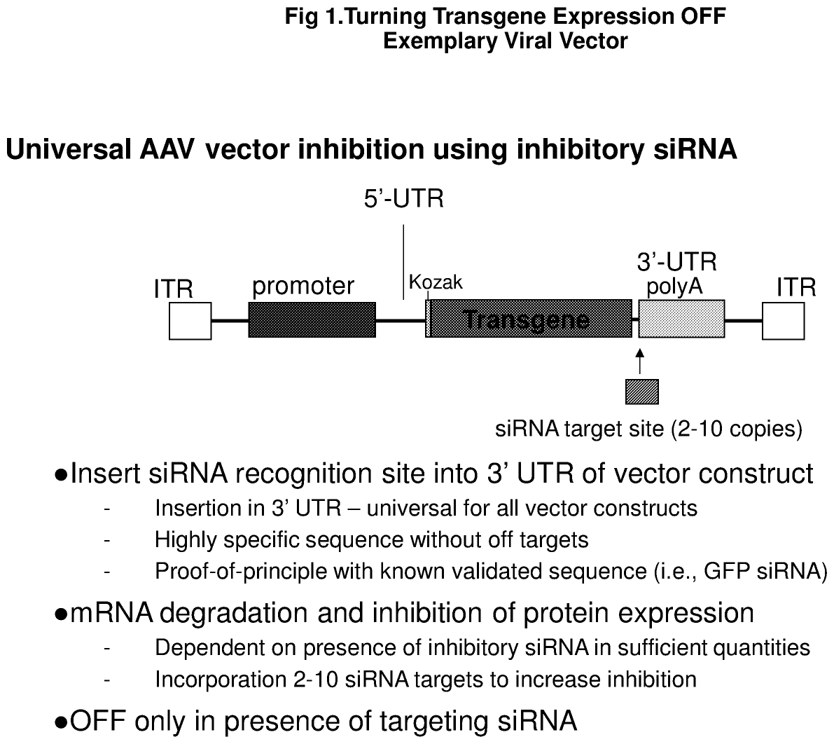

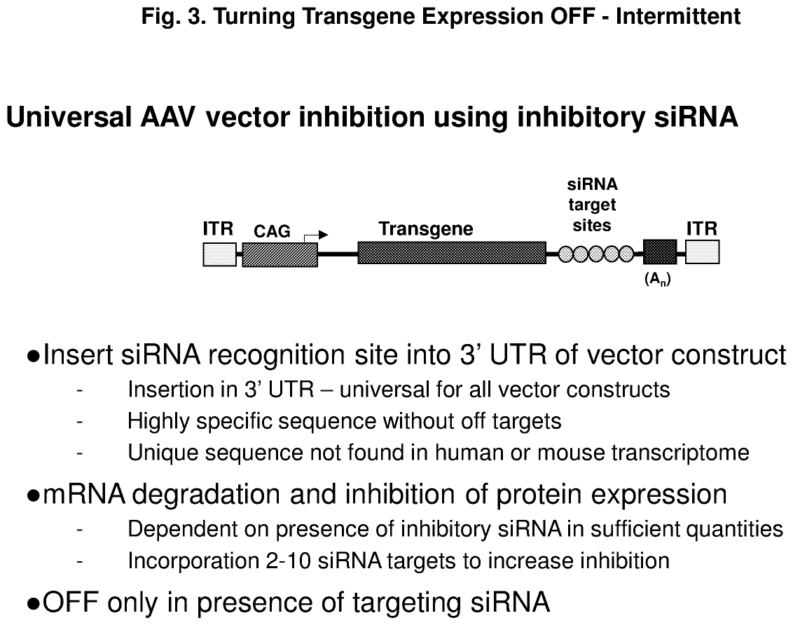

FIGS. 1 and 3 show vectors for turning expression off by inserting a siRNA recognition site into a 3′ UTR of vector construct. In one embodiment, the insertion into the 3′ UTR is a sequence that is universal for all vector constructs, e.g., a highly specific sequence without off targets. mRNA degradation and inhibition of protein expression is dependent on presence of inhibitory siRNA in sufficient quantities. In one embodiment, incorporation of from, for example, 2 to 10 copies of one or more siRNA target sequences may increase inhibition. Expression is turned off only in presence of targeting siRNA.

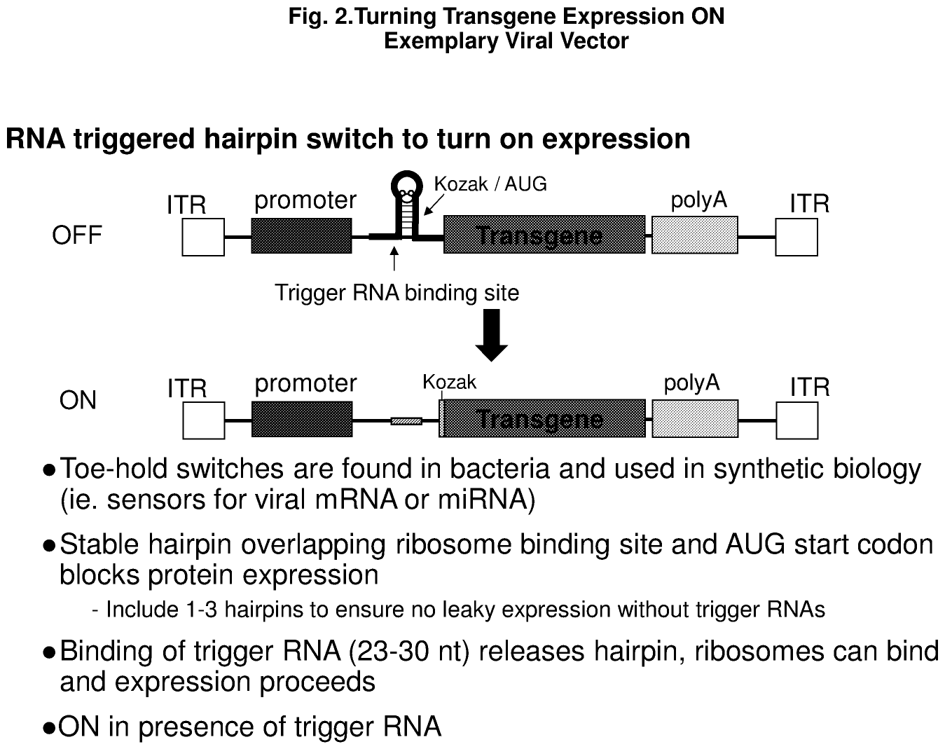

FIG. 2 illustrates vectors for turning expression on. For example, toe-hold switches are found in bacteria and used in synthetic biology (e.g., as sensors for viral mRNA or miRNA). A stable hairpin overlapping a ribosome binding site and/or AUG start codon blocks protein expression. Inclusion of from, for example, 1 to 3, hairpins may ensure no leaky expression without trigger RNAs. Binding of trigger RNA (e.g., having a length from about 23 to 30 nt) releases the hairpin(s), so that ribosomes can bind and expression proceeds. Thus, expression is “on” in presence of trigger RNA.

Thus, gene therapy vectors are provided with transgene expression levels that can be modulated by the application of exogenous small RNAs. Transgene expression from a gene therapy vector can be turned up or down with application of exogenous small RNA. RNA recognition sequences are built in to 5′ and/or 3′ UTR of vector transgene product and may be universal for all transgenes. Benefits of tunable transgene expression includes turning on expression for, for example, only a limited time or turning off expression for, for example, enhanced safety or lower toxicity, as well as use of a combination of an on switch and an off switch in the same vector.

### Example 2

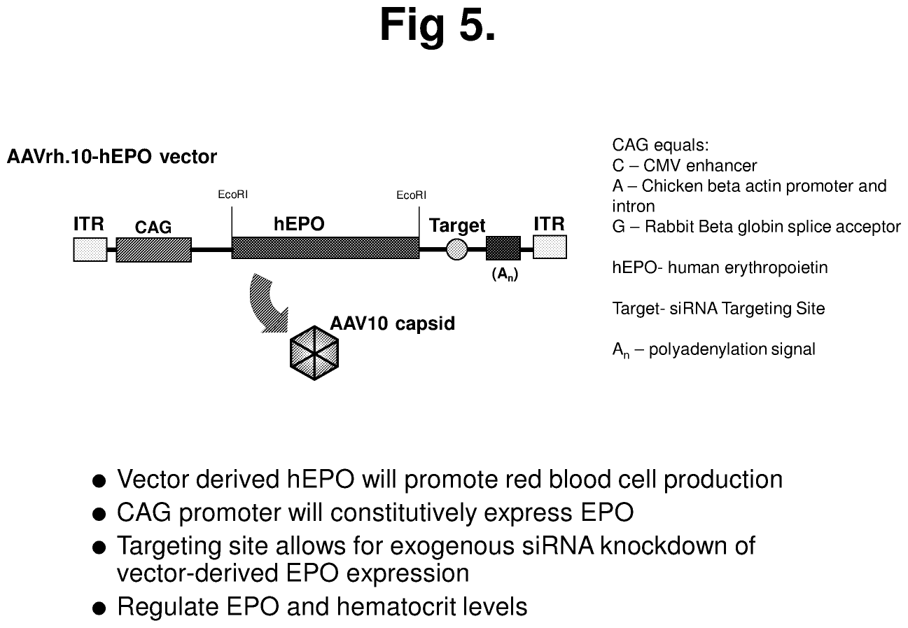

FIG. 5 shows a vector for regulatable EPO expression. Vector derived hEPO promotes red blood cell production and the CAG promoter constitutively expresses EPO. The targeting site allows for exogenous siRNA knockdown of vector-derived EPO expression, thereby providing regulation of EPO and hematocrit levels.

Exemplary target sites include but are not limited to: siRNA having seed sequences that score better than 99.99% and 99.67% in the siSPOTR algorithm and only match, for example, 12 to 13/21 nucleotides in either the human or mouse genome.

Exemplary target sequences in the vector are shown below:

Target 1:
(SEQ ID NO: 1)
AAGATCGTCGTAAGCGCGTAA
Target 2:
(SEQ ID NO: 2)
AAGTTACGCGACTTACGCGAT
Target 3:
(SEQ ID NO: 3)
ACGATCGTTCATATCGCGTAT

Exemplary siRNAs to be supplied in trans for targets 1-3. e.g., are as follows:

siRNA 1:
(SEQ ID NO: 4)
GAUCGUCGUAAGCGCGUAAuu (SENSE)
siRNA 2:
(SEQ ID NO: 5)
GUUACGCGACUUACGCGAUuu (SENSE)
siRNA 3:
(SEQ ID NO: 6)
GAUCGUUCAUAUCGCGUAUuu (SENSE)

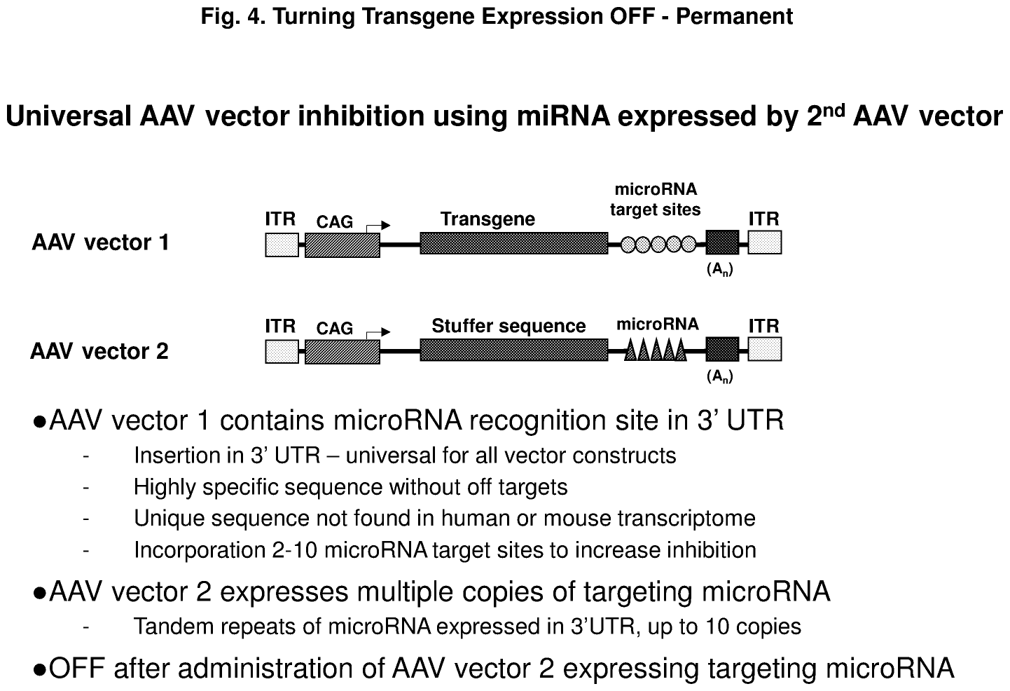

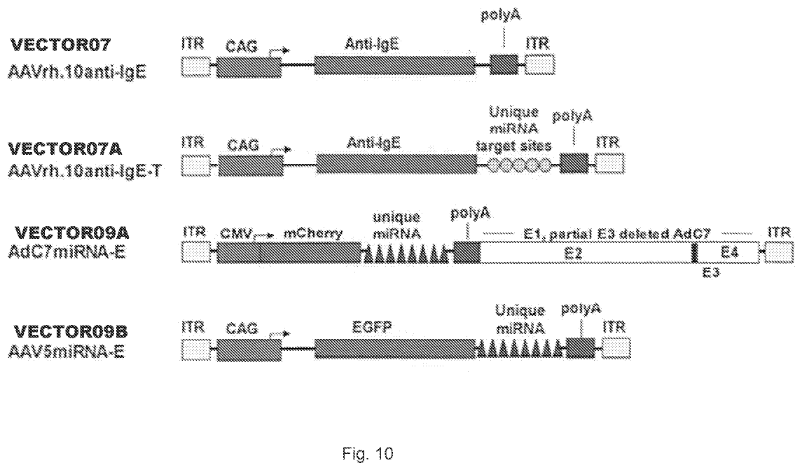

Exemplary targets 4-6 may be employed in a system that employs miRNA, e.g., for the AAV/Ad expressed miRNA (see, e.g., FIG. 4 and FIG. 10).

Target 4:
(SEQ ID NO: 7)
CTATTGACGTATGACGCGTAA
Target 5:
(SEQ ID NO: 8)
CAATCCATCATTACGCGTTAA
Target 6:
(SEQ ID NO: 9)
CAATCCATCAATTACGCGTTA

For the purposes of the ON switch (FIG. 2) exemplary sequences to trigger the hairpin may be the same as the siRNAs for the inhibition (Targets 1-3, siRNA 1-3).

Two potential negative control siRNAs were designed using the same criteria (excluded any with seed sequence similar to targeting siRNAs):

Control siRNA 1:
(SEQ ID NO: 10)
AAGATCCCGATTGTCGATCGT
Control siRNA 2:
(SEQ ID NO: 11)
ACGTCGTACGTTACCGTACGA

### Example 3

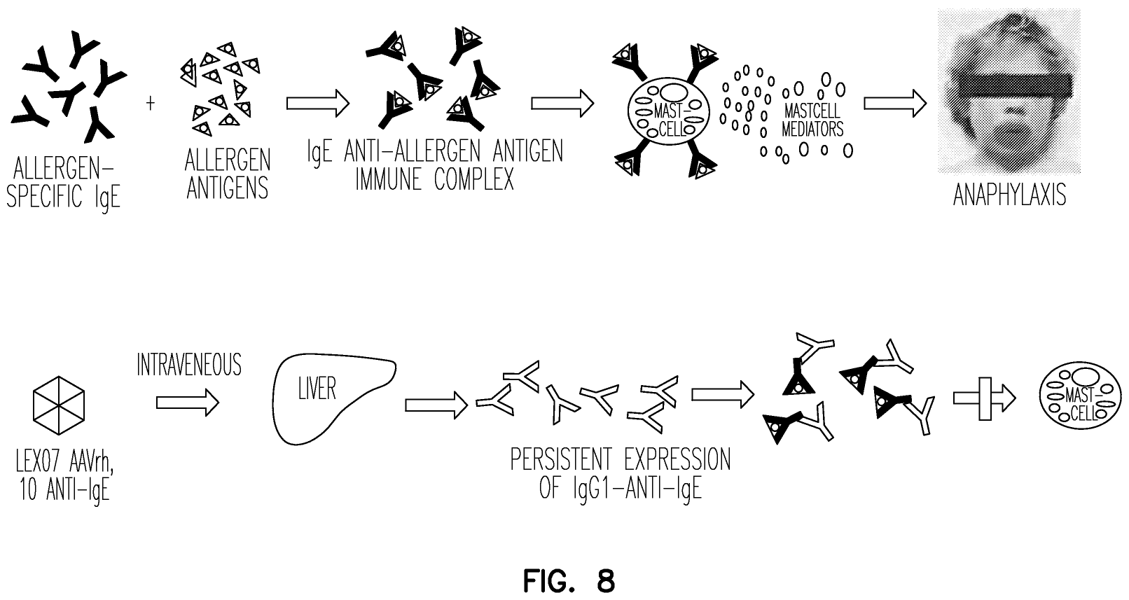

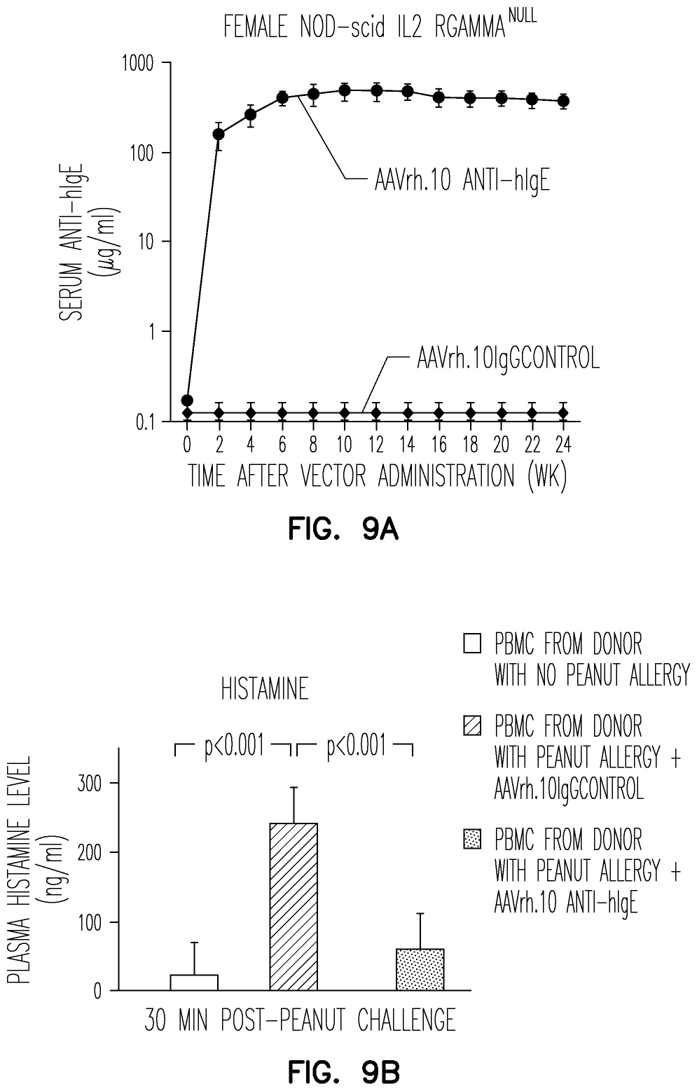

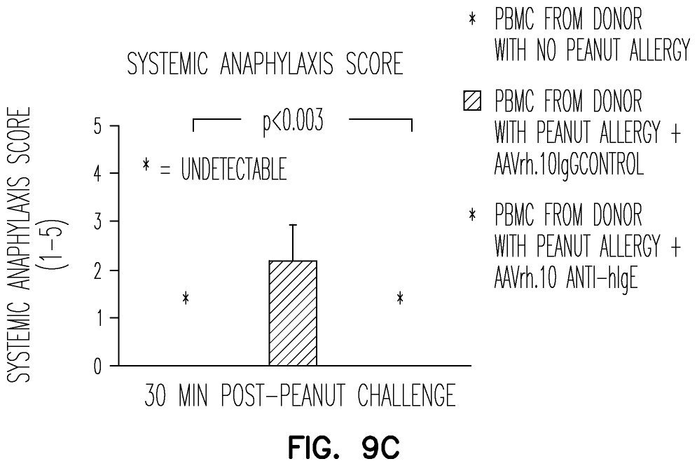

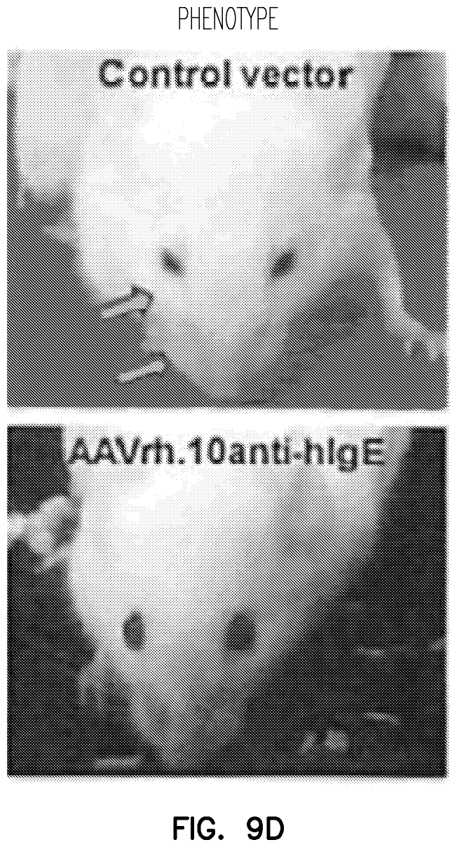

Vectors were prepared for a treatment to prevent severe reactions (anaphylaxis) mediated by type I hypersensitivity responses linked to allergen-specific immunoglobin E (IgE) (Pate et al., 2010; Burton & Oettgen, 2011; Wu & Zarrin, 2014) (FIG. 8). The IgE-allergen complex activates mast cells and basophils, with release of mediators responsible for anaphylaxis response to the allergen (Pate et al., 2010; Burton & Oettgen, 2011; Wu & Zarrin, 2014). The current therapy to interrupt allergen-specific IgE from eliciting anaphylaxis is omalizumab (Xolair®), a recombinant DNA-derived humanized IgG1K monoclonal antibody that binds to human immunoglobulin E (IgE) (Babu et al., 2013), inhibiting the binding of IgE to IgE receptor (FcεRI) on the surface of mast cells and basophils, suppressing the release of mediators of the allergic response from FcεRI hearing cells (Holgate et al., 2005). Once circulating IgE antibodies are bound by anti-IgE, they cannot bind to specific high affinity Fc receptors, and the complexes of anti-IgE bound to IgE are cleared from the circulation (Babu e al., 2013). While omalizumab is effective (Babu e al., 2013; Leung et al., 2003; Schneider et al., 2013; Shikh & Burks, 2013), using it as prophylaxis has challenges as the protection provided by a single administration is short (24 weeks), requiring monthly parenteral administration to maintain persistent effective therapy (Lowe et al., 2009: Lieberman & Chehade, 2013). As a strategy to bypass the requirement for repeated administration, vector07, where a single administration of a serotype rh.10 adeno-associated virus (AAV) coding for omalizumab (AAVrh.10anti-IgE) provides long term expression of anti-IgE, protecting against allergen-specific-induced allergic reactions. A humanized mouse model of human IgE-mediated anaphylaxis in response to peanuts was created using NOD-scid IL2Rgamma^{null} immunodeficient mice reconstituted with peanut-specific human blood mononuclear cells (Pagovich et al., 2016). In these mice, total and peanut-specific IgE levels were generated after engraftment of mononuclear cells from peanut allergic individuals, and peanut-specific anaphylaxis was induced by the release of mediators after challenge with peanut extract. With a single administration of vector07, there was persistent prevention of peanut-induced severe allergy, either when administered as a prophylaxis before peanut sensitization or as a therapy after the mice exhibit the peanut-induced anaphylaxis-related symptoms (FIG. 9).

In one embodiment, the present disclosure provides for vector, having “off switches” to shut off the expression of, for example, the anti-IgE acutely (to deal with, e.g., anaphylaxis) and chronically (to deal with, e.g., parasitic infection). The “off switch” strategy is based on embedding in the therapeutic expression cassette a sequence responsive to microRNA (miRNA) that shuts down the anti-IgE expression directed by vector07. To enable both rapid and persistent shutdown of anti-IgE expression, vector07 was modified to vector07A to include 5 tandem repeats of a 21 bp miRNA target sequence 3′ to the anti-IgE coding sequence (vector7A, AAVrh.10hanti-IgE-T; FIG. 10). The design of vector07A, and the cognate miRNA delivery vectors [vector09A and vector09B (FIG. 10)] are based on the identification of a unique 21 bp miRNA target site not present in the human or mouse genome using on the algorithms of Boudreau et al. (2013) and Birmingham et al. (2007); this unique target has >99.99% probability of no off target effects. In one embodiment, to shut down vector07A-mediated expression of anti-IgE, 2 gene transfer “off” vectors were designed. Following IV administration, these vectors target the liver and express the effector miRNA that binds to the cognate target in the vector07A-generated mRNA, identifying the mRNA for destruction, shutting down of the vector07A-mediated expression of anti-IgE. To rapidly shut down anti-IgE expression from vector07A, vector09A (AdC7miRNA-E), a serotype C7 adenovirus (AdC7) vector coding for the miRNA cognate to the vector07A miRNA target, is employed. AdC7 is effective despite the immunity evoked by vector07A as there is no cross-reactivity between Ad and AAV vectors (Blacklow et al., 1967; McCaffrey et al., 2008; De et al., 2008) since there are no homologous epitopes, and AdC7 is derived from a chimpanzee for which the human population has no preexisting immunity (Zhi et al., 2006; Roy et al., 2004; Basnight et al., 1971; Reyes-Sandoval et al., 2004). Based on the known pharmacokinetics of Ad vectors, following IV administration, vector09A expresses the cognate miRNA in the liver within 8 hours, thus rapidly shutting down the vector07A-mediated hepatocyte expression of anti-IgE (McCaffrey et al., 2008; De et al., 2008; Wen et al., 2000). To permanently shut down the anti-IgE expression from vector07A (AAVrh.10anti-IgE-T), vector09B (AAVSmiRNA-E), a serotype 5 AAV vector that functions effectively to express the effector miRNA, despite the anti-AAVrh.10 immunity evoked by the prior administration of vector7A (an AAVrh.10 vector) (De et al., 2006), is employed. The capsids of AAVrh.10 and AAV5 are derived from sufficiently distinct clades that prior immunity to AAVrh.10 does not interfere with normal expression from the AAV5-based vector (vector09B) used to shut down the anti-IgE expression from vector07A (Sondhi et al., 2007; Gao et al., 2004; Piguet et al., 2012). Finally, the combination of vector09A and vector09B together acutely and chronically shuts down vector07A, e.g., permanently shuts down anti-IgE expression indefinitely starting 8 hours after administration.

In one embodiment, a rAAV comprises a rAAV genome encoding a gene of interest. e.g., an antibody such as anti-IgE or anti-Siglec, comprising AAV8, AAV9, AAV5, AAV2, or AAVrh10 capsid. The rAAV genome may be of any AAV serotype including but not limited to an AAV8, AAV9. AAV5, AAV2, or AAVrh10 genome. In one embodiment, a second viral vector expresses siRNA in the form of shRNA, or saRNA (trigger RNA) as miRNA. In one embodiment, after administering a rAAV comprising an AAV8, AAV9, AAV5, AAV2, or AAVrh10 capsid, a rAAV4 or rAAV5 may be administered. In one embodiment, after administering a rAAV, a non-AAV viral vector is administered, e.g., an adenovirus, lentivirus, herpesvirus or retrovirus vector.

In one embodiment, a recombinant adenovirus, lentivirus, herpesvirus or retrovirus encoding a gene of interest is employed. In one embodiment, a second viral vector expresses siRNA in the form of shRNA, or saRNA as miRNA. In one embodiment, after administering a recombinant adenovirus, lentivirus, herpesvirus or retrovirus vector, a second heterologous viral vector, e.g., a rAAV8, rAAV9, rAAV5, rAAV2, or rAAVrh10 vector, may be administered.

### Example 4

### Introduction

Allergens account for a significant number of fatal and near-fatal, anaphylactic reactions in the US (Sheikh & Burks, 2013; Liu et al., 2010). With exposure to an allergen to which they are sensitized, individuals with severe allergies manifest itchiness, urticaria, swelling, eczema, airway constriction, abdominal pain, hypotension and anaphylaxis (Simons et al., 2011; Taylor et al., 2010). Allergen-induced anaphylaxis includes vomiting, diarrhea, abdominal pain, angioedema, laryngeal edema, bronchospasm, lower-airway obstruction, hypotension, loss of consciousness and sometimes death (Simons et al., 2011; Taylor et al., 2010). Most allergies start in childhood and many are not outgrown (Sheikh & Burks, 2013; Sampson, 2013). Affected individuals must strictly avoid the allergen and have quick access to an epinephrine auto-injector (Schneider et al., 2013; Simons et al., 2011; Du et al., 2015; Sicherer et al., 2010). The only available therapies are acute use of epinephrine, desensitization or anti-IgE monoclonals. The anti-IgE monoclonal omalizumab is effective but there are a number of reasons why vector7A would be preferable, including: (1) anti-IgE monoclonals have to be administered every 2 to 4 weeks (Lowe et al., 2009); therapy with vector07A is a single administration; (2) systemic administration of an anti-IgE monoclonal results in immediate high anti-IgE levels, but then descending levels, providing less protection over time; in contrast, serum anti-IgE levels expressed by vector07A are constant, a more optimal pharmacokinetic property; and (3) the requirement for an anti-IgE monoclonal to be given on a repetitive basis adds to cost of care, requires repetitive parenteral administration by a health provider and is inconvenient for the individual being treated.

### Exemplary Vector and Method

One-time therapy with vector07A would obviate the risk for repetitive severe allergic reactions. To ensure safety of vector07A, an “off switch” for AAV-mediated gene therapy is employed, a strategy that is useful not only for vector07A anti-IgE gene therapy, but as a platform that can be used to shut down any gene therapy with the miRNA target built into the expression cassette.

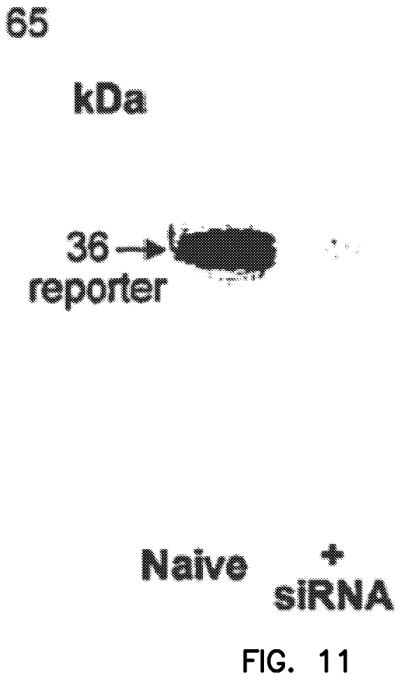

Vector07A (AAVrh.10anti-IgE-T) is a nonhuman primate serotype rh.10 AAV vector expressing omalizumab under control of the CAG highly active constitutive promoter. It is identical to vector07 but includes (3′ of the omalizumab anti-IgE heavy and light chain coding sequence) 5 tandem repeats of 21 bp sequence that serve as the target for the miRNA expressed by vector09A and vector09B (FIG. 11). The 21 bp targets and its cognate miRNA are unique sequences not present in the mouse or human genome. Following IV administration, >90% of vector07A is expressed in the liver, an organ highly effective for expression and secretion of gene therapy products mediated by AAV vectors (Pagovich et al., 2016; Mingozzi & High, 2011; Sands, 2011; van der Laan et al., 2011). The safety of administration of AAVrh.10 to experimental animals (Piguet et al., Rosenberg et al., 2018; Sondhi et al., 2012; Chiuchiolo et al., 2013; Rosenberg et al., 2014; Zerah et al., 2015) and humans has been demonstrated (BB-IND1539). Omalizumab is an FDA approved anti-IgE IgG1k monoclonal (trade name Xolair®, no longer under patent, US2001/6329509) that specifically binds to free IgE and membrane bound IgE on the surface of lymphocytes (Chang et al., 2007; Schulman, 2001). Vector07A prospective patients are first tested with omalizumab to determine if there is risk of anaphylaxis, which most often occurs—with the 1st administration (Lieberman & Chehade, 2013; Kim et al., 2010). As an additional safety strategy, 2 vector07A “off switches” that mediate rapid and persistent shut down of the therapeutic AAVrh.10 vector mediating persistent liver hepatocyte expression of anti-IgE are prepared. Vector07A has an embedded miRNA target sequence that when based by a cognate miRNA, shut down expression of the anti-IgE if necessary for safety issues (FIG. 11). Vector09A, a serotype C7 Ad coding for the cognate miRNA that, when administered IV, expresses in liver hepatocytes and rapidly shuts down anti-IgE expression mediated by vector7A. Vector09B, a serotype 5 MV coding for the same cognate miRNA that when administered IV, expresses in liver hepatocytes and persistently shut downs anti-IgE expression. Vector09A and vector09B are not product specific; they can be used for any gene therapy-based therapeutic strategy for which the ability to shut off expression is an important safety feature, as long as the miRNA target sequence is built into the therapeutic vector.

15 million people in the US have food allergies (Branum & Lukacs, 2010; Gupta, 2011; Liu et al., 2010). Allergies to peanuts, tree nuts, fish and shellfish are generally lifelong (Skripak et al., 2007: Savage et al., 2010; Savage et al., 2007; Keet et al., 2009; Sicherer et al., 2004). For children, food allergies result in 300,000 ambulatory care units/yr and 9,500 hospitalizations/yr ((Branum & Lukacs, 2010). There are 200,000 emergency department visits for food allergy reactions/yr and food allergy is the leading cause of anaphylaxis (Sampson, 2003; Clark et al., 2011). Insect sting allergies affect 5% of the US population, with 90-100 deaths/yr due to insect sting anaphylaxis. An advantage of vector07A is that it will markedly reduce severe allergic episodes, with a one-time therapy.

Thus, vector07A gene therapy would require only a single administration to provide sustained protection from anaphylaxis due to allergen challenges; this can be applied to any IgE-mediated allergy. The development of vector07A with the off switch targeting sequence and rapid (vector09A) and persistent (vector09B) “off switches,” are generic for AAV drug development.

Preliminary studies. The efficacy of vector07 was demonstrated in a humanized model of peanut allergy. Vector07A is identical to vector07 except for additional miRNA off target sequences 3′ to the anti-IgE sequence. To demonstrate the function of this unique 21 bp miRNA target in vector07A, a plasmid was designed with the same promoter (CAG) as vector07A, but with a reporter gene, followed 3′ by the effector mi RNA (miRNA-E) derived from a modified mir155-based backbone (Fowler et al., 2016) in which the guide strand was replaced by sequence complementary to the unique kDa targeting site in vector07A. Transfection of the plasmid into 293T cells± an siRNA (identical to the sequence of the miRNA expressed by vector09A and vector09B), demonstrated >99% suppression of the mRNA expression of the reporter gene (FIG. 11).

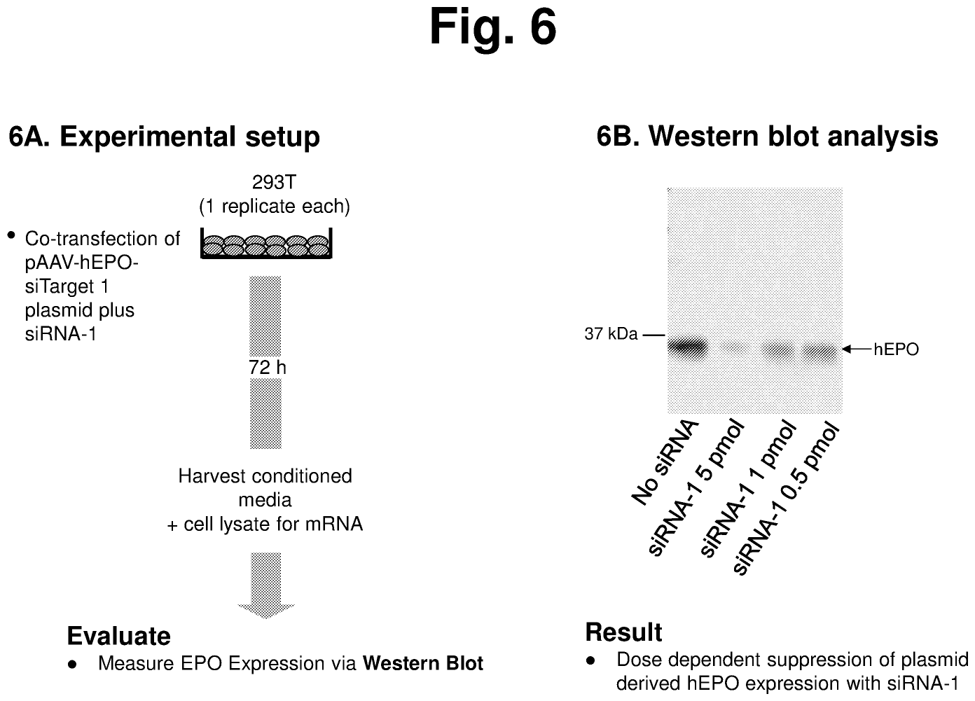

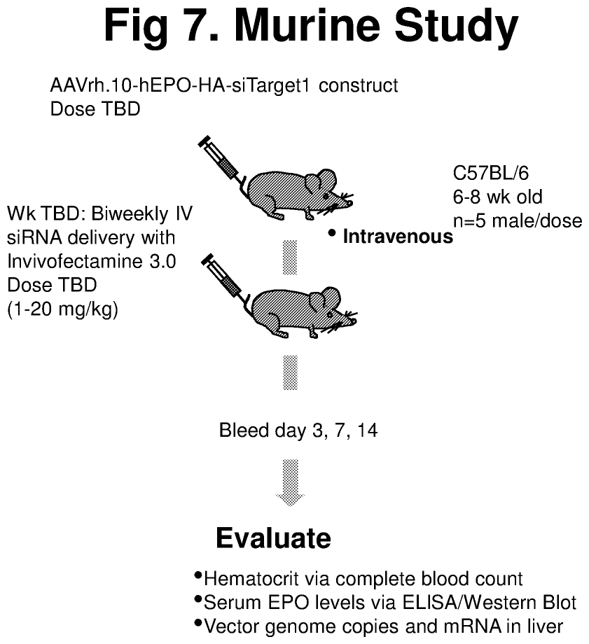

AdC7. Vector09A (AdC7miRNA-E; FIG. 10) is based on a chimpanzee AdC7 vector (Roy et al., 2004; Krause et al., 2013; Zhi et al., 2005). AdC7 is not recognized by anti-AAV neutralizing antibodies. Construction and production of AdC7 vectors is as described in Zhi et al. (2005) and Worgall et al. (2005). AAV5. Vector09B (AAVSmiRNA-E; FIG. 10) is constructed and produced as in De et al. (De et al., 20060. Efficacy of AAV5 in the context of anti-AAVrh.10 immunity. One challenge of in vivo gene therapy is that administration of an AAV vector induces neutralizing immunity to the AAV capsid, such that repeat administration of the same or similar capsid is ineffective: the neutralizing anti-capsid immunity prevents the capsid of the repeat administration from reaching its cognate receptors. This can be circumvented by seroswitch, i.e., using capsids of different clades. This approach has been successful for Ad71 and AAV vectors (Gao et al., 2002; Davidoff et al., 2005), including the demonstration of efficient expression with an AAV5 vector in the context of AAVrh.10 neutralizing immunity (see FIG. 6 in De et al. (2006).

Combined administration of Ad and AAV vectors to mediate rapid and sustained expression. While AAV vectors provide sustained expression following a single administration, the onset of expression is about 1 week, insufficient to provide acute therapy necessary to shut off vector07A anti-IgE expression in the context of anti-anti-IgE induced anaphylaxis. In contrast, the onset of expression of Ad-based vectors is 8 hours (McCaffrey et al., 2008; Wen et al., 2000; Chu et al., 2019; Greenberg et al., 2020). To provide rapid and sustained shut off of vector07A, combined administration of vector09A (AdC7) and vector09B (AAV5) is employed to provide rapid, sustained expression of miRNA-E. As discussed above, Ad vectors are not seen by immunity evoked by AAV vectors. AAV5 vectors effectively express their transgene in the context of anti-AAVrh.10 immunity (De et al., 2006). Combined administration of Ad and AAV vectors provides rapid and sustained expression of the same transgene (De et al., 2008).

Studies. Vector07 is altered to vector07A to include a target miRNA that responds to AdC7 (rapid) and/or AAV5 (persistent) “off switches” which shut down vector07A-mediated liver hepatocyte expression of anti-IgE. An overview of the studies are in Table I followed by the details of the studies.

Testing of vector07A. Regarding the properties of vector07A and vector07, following IV administration (2 doses each), mice are assessed at 4 and 8 weeks for equivalence (±20%) of liver anti-IgE mRNA and serum anti-IgE protein levels (see Pagovich et al. (2016) for details).

Testing of vector09A and vector09B. Vector09A and vector09B are tested for liver expression of the effector miRNA by TaqMan quantification of liver homogenate. Assessments are made at 1 day, 1, 4 and 8 weeks after administration. Based on Ad and AAV vectors, vector09A (AdC7 mCherry-miRNA-E) expresses in day 1 (8 hours), peak at day 7 and be <5% peak by 4 wk. Vector09B (AAV5EGFP-miRNA-E) is 75% expressed by week 2, 100% by weeks 3-4, and thereafter persistently expressed at the same level.

Vector shut off (shut down of vector07 A-mediated expression of anti-IgE). Four weeks after vector07A administration, when levels of anti-IgE mRNA (liver) and protein (serum) have stabilized (Pagovich et al., 2016), studies are carried out to shut down vector07A expression, either or both of the acute off vector (vector09A) and persistent off (vector09) is/are administered IV. The assessment of “off” success includes: liver anti-IgE mRNA (TaqMan) and serum anti-IgE protein (ELISA)<10% of vector07A alone. To verify that the vector09A miRNA product is expressed in the same liver cells as the vector07A anti-IgE product, the liver at each time point is assessed for vector07A mRNA (in situ hybridization) (Chu et al., 2019; Greenberg et al., 2020) and the marker genes (mCherry or EGFP) expressed by the vector09A (mCherry) or vector09B (EGFP; both assessed by immunofluorescence) (Fe et al., 2017).

Vector09A (AdC7-based) and vector09B (AA V9-based) “off” vectors were designed to function in the context of the anti-AAVrh.10 immunity evoked by the administration of vector07A, the therapeutic vector (based on AAVrh.10). To assess immunity evoked by all of the vectors, all time points are assessed for neutralizing antibodies against AAVrh.10, AdC7 and AAV5 (De et al., 2006; Rosenberg et al., 2018; Sondhi et al., 2012; Wang et al., 2014).

Detailed methods. The vector07, 7A and 9A AAVrh.10 vectors and the vector09A AdC7 vector are manufactured, purified and tested as previously described (De et al., 2008; De et al., 2006; Sondhi et al., 2007; Rosenberg et al., 2018; Krause et al., 2011). For liver anti-IgE mRNA (TaqMan) and serum anti-IgE (ELISA), see Pagovich et al. (2016). Liver hepatocyte co-expression of transgenes are assessed by anti-IgE in situ hybridization, mCherry and EGFP immunofluorescence (Fe et al., 2017). Serum neutralizing antibodies against AAVrh.10, AdC7 and AAV5 are assessed with capsid-marker gene expression in vitro (De et al., 2006; Rosenberg et al., 2018).

TABLE I
Murine Studies to Test vector07A, vector09A and vector09B¹

| | | | | | Time of evaluation⁴ | | |
|---|---|---|---|---|---|---|---|
| | Vector administration² | | | | After 1st | After 2nd | |
| Aim | 0 time | At 4 wk | Doses³ | n⁴ | vector | vector | Parameters⁵ |
| 1 | Vector07 | — | \| | | 4, 8 wk | — | Liver anti-IgE |
| | Vector07A | — | 10¹⁰, | | 4, 8 wk | — | mRNA; serum anti- |
| 2 | Vector09A | — | 10¹¹\| | 5M, | 1 d; 1, 4, 8 wk | — | IgE protein; liver |
| | Vector09B | — | \| | 5F | 1 d; 1, 4, 8 wk | — | miRNA-E; hepato- |
| 3 | Vector07A PBS | | Optimal | each | 4 wk | 1 d; 1, 4, | cyte mCherry |
| | | | dose | dose, | | 8 wk | EGFP, in situ anti- |
| | Vector07A Vector09A | | from | each | 4 wk | 1 d; 1, 4, | IgE; serum neutraliz- |
| | | | aim 1 | time | | 8 wk | ing antibodies |
| | Vector07A Vector09B | | | point | 4 wk | 1 d; 1, 4, | AAVrh.10, AAV5, |
| | | | | | | 8 wk | Adc7 |
| | Vector07A Vector09A + | | | | 4 wk | 1 d; 1, 4, | |
| | Vector09B | | | | | 8 wk | |
| 1-3 | PBS PBS | | | | Pre, 4 wk | 1 d; 1, 4, | |
| | | | | | | 8 wk | |

All studies in CS7B1/6 mice, age 6-8 wk; PBS (phosphate buffered saline) control for all 3 aims; ²See FIG. 10 for vector description; vectors or PBS administered in 100 μl IV via tail vein; ³Doses for Vector07, Vector07A and Vector09B (all AAV vectors) are in genome copies; doses for Vector09A (Ad vector) is in particle unites; for aims 1, 2, 2 doses will be tested; for aim 3, the optimal dose for aims 1, 2 will be used; ⁴For each time point, each dose and each vector (or PBS), n=5 male, 5 females. For aim 1 time of assessment 4, 8 weeks after administration; for aim 2, 1 d, 1, 4, 8 wk after administration; for aim 3, 4 weeks after Vector07, then 1 d, 1, 4, 8 wk, after 2nd vector; for PBS control, pre; 4 wk after 1st administration 1 d; 4, 8 wk. The “1 day” assessment for Vector09A “off” will be at 8 hr (the AdC7 vector expresses at 8 hours and peaks at 7 d); ⁵Aim 1—liver anti-IgE mRNA, serum anti-IgE protein, serum neutralizing antibodies: aim 2—liver miRNA-E, serum neutralizing antibodies: aim 3—all parameters; PBS—all parameters.

Statistics. The statistical analyses are performed using repeated measures ANOVAs applied separately to each assay (see Table I) with factors for dose, sex, and a repeated measure for time of evaluation. Appropriate pre-hoc contrasts are then considered to assess the central hypotheses. For example, one study, that contrasts within 4 and 8 wk and for both combined (for anti-IgE mRNA, protein, in situ anti-IgE, and antibodies), is used to identify if there are any significant differences between vector07A and vector07, and for significant differences of each compared to PBS, as well as to estimate any differential impacts of dose, similar approach is taken (e.g., for assays miRNA-E, hepatocyte mCherry, EGFP) to assess differences in dose and to assess hypotheses for expected change over time (e.g., expected levels of vector09A significantly higher than vector09B at 1 d and 1 wk and the reverse for 4 and 8 wk). For another study, appropriate contrasts are used to assess differences compared to PBS and to compare before/after 2nd vector evaluation at the different time points to assess significant changes in anti-IgE assays for vector09A (e.g., expected to be high at 1 d, 1 wk with vector07A “off”), vector09B (high at 4, 8 wk with vector07 A “off”) and vector09A+ vector09B (high at all 2nd vector time points/vector07A “off”). Given anti-IgE levels from vector07 administration (e.g., see FIG. 9) and assuming similar study vector expression, the sample size (e.g., 5 M/F mice per dose, per time point) makes these contrasts well-powered to detect significant impacts of treatment.

The number of repeats may be reduced from 8 to 6 or 4 or 2. If shut down of vector07A is not complete, an additional unique target and effector miRNA combination could be added to vector07A and vector09A, vector09B, respectively.

Mice are treated as adults, 8 wk of age, and assessed for 4 and 8 wk, and 1 day, 1, 4, and 8 wk, and at several time-points post-treatment with AAV and/or Ad. The AAVrh.10, AAV5, and AdC7 may be tested at 2 doses (1×10^{1Q} and 1×10¹¹ gc), and then with one dose. For example, 2 vectors (Vector07, vector07A)×2 doses×10 mice (5M/5F)/dose×2 time points, are used; 2 vectors (Vector09A, vector 09B)×2 doses×10 mice (5M/5F)/dose×4 time points are used; or 4 treatment groups×1 dose×5 time points×10 mice (5M/5F)/time, are used; plus PBS control groups.

Administration of AAV and Ad vectors will involve a non-surgical procedure of intravenous delivery route via tail veins to the mice. Briefly, mice are carefully warmed (with a heat lamp) to cause venodilation, increasing ease of vascular access. The mouse is placed in a restraining device such that the lateral tail veins are accessible. The tail is cleansed with a sterile alcohol wipe prior to injection. An insulin 28G needle is directed into a lateral tail vein, and the injectate (AA V, Ad. or PBS) is slowly administered, making sure that no swelling is detected cranial to the injection site. Pressure is applied over the injection site after the needle is withdrawn from the vein for approximately 30 seconds with gauze (or similar material) to prevent hematoma formation and make sure that hemostasis is achieved.

Exemplary sequences for target sites and miRNAs for vectors described in FIG. 4 and FIG. 10 are as follows where the miRNAs are derived from the modified mir155 scaffold described in Fowler et al. (2016).

miRNA1:
(SEQ ID NO: 12)
CTGGAGGCTTGCTTTGGGCTGTATGCTGTTACGCGTCATACGTCAATAG
GTTTTGGCCACTGACTCGACTTATGATTATGACGCGTAACAGGACACAA
GGCCCTTTATCAGCACTCACATGGAACAAATGGCCACCGTGGGAGGATG
ACAA
miRNA2:
(SEQ ID NO: 13)
CTGGAGGCTTGCTTTGGGCTGTATGCTGTTACGCGTCATACGTCAATAG
GTTTTGGCCACTGACTCGACTTATTATGATGACGCGTAACAGGACACAA
GGCCCTTTATCAGCACTCACATGGAACAAATGGCCACCGTGGGAGGATG
ACAA
miRNA3:
(SEQ ID NO: 14)
CTGGAGGCTTGCTTTGGGCTGTATGCTGTTAACGCGTAATGATGGATTG
GTTTTGGCCACTGACTCGACTAATTATATTACGCGTTAACAGGACACAA
GGCCCTTTATCAGCACTCACATGGAACAAATGGCCACCGTGGGAGGATG
ACAA
miRNA4:
(SEQ ID NO: 15)
CTGGAGGCTTGCTTTGGGCTGTATGCTGTTAACGCGTAATGATGGATTG
GTTTTGGCCACTGACTCGACTAATCATTTTACGCGTTAACAGGACACAA
GGCCCTTTATCAGCACTCACATGGAACAAATGGCCACCGTGGGAGGATG
ACAA
miRNA5:
(SEQ ID NO: 15)
CTGGAGGCTTGCTTTGGGCTGTATGCTGTAACGCGTAATTGATGGATTG
GTTTTGGCCACTGACTCGACTAATTATAATTACGCGTTACAGGACACAA
GGCCCTTTATCAGCACTCACATGGAACAAATGGCCACCGTGGGAGGATG
ACAA
miRNA6:
(SEQ ID NO: 16)
CTGGAGGCTTGCTTTGGGCTGTATGCTGTAACGCGTAATTGATGGATTG
GTTTTGGCCACTGACTCGACTAATCATTATTACGCGTTACAGGACACAA
GGCCCTTTATCAGCACTCACATGGAACAAATGGCCACCGTGGGAGGATG
ACAA

Exemplary sequences for the hairpins for the ON switch shown in FIG. 2 are as follows:

Hairpin 1A
(SEQ ID NO: 17)
AAAAGATCGTCGTAAGCGCGTAATTTTCCATCAAGAACAGGCCACCATG
GAAAATTACGCGAACCTGGCGGCAGCGCAAAAGATGGGGGTGCACGAAT
GTCCTGCC
Hairpin 1B
(SEQ ID NO: 18)
AAAAGATCGTCGTAAGCGCGTAATTTTCCATCTAGAAGACGCCACCATG
GAAAATTACGCGAACCTAGCCCCAGCCCAAAAGATGGGGGTGCACGAAT
GTCCTGCC
Hairpin 2A
(SEQ ID NO: 19)
AAAAGTTACGCGACTTACGCGATTTTTCCATCAAGAACAGGCCACCATG
GAAAAATCGCGTAACCTGGCGGCAGCGCAAAAGATGGGGGTGCACGAAT
GTCCTGCC
Hairpin 2B
(SEQ ID NO: 20)
AAAAGTTACGCGACTTACGCGATTTTTCCATCAAGAAGACGCCACCATG
GAAAAATCGCGTTACCTAGCCCCAGCCCAAAAGATGGGGGTGCACGAAT
GTCCTGCC.

The corresponding triggers are siRNA #1 and siRNA #2.

### REFERENCES

Araujo et al., Mem. Inst. Oswaldo Cruz, 101:925 (2006).
Bachour & Bennett. J. Assoc. Genet. Technol., 32:21 (2011).
Back et al., Infect. Ecol. Epidemiol., 3:19839 (2013).
Beisel et al., Nucleic Acids Res., 39:2981 (2011).
Bharaj et al., Virol. J., 5:1 (2008).
Bhatt et al., Nature, 496:504 (2013).
Bouzid et al., BMC Public Health, 14:781 (2014).
Cambray et al., Nucleic Acids Res., 41:5139 (2013).
Carothers et al., Science, 334:1716 (2011).
Chan et al., Anal. Methods, 10, 214 (1018).
Chan et al., SLAS Discov., 24:68 (2019).
Chang et al., Curr. Opin. Biotechnol., 23:679 (2012).
Chappell et al., Nat Chem. Biol., 11:214 (2015).
Chatterjee et al., ACS Synth. Biol., 2:2737 (2018).
Chen et al., Proc. Natl. Acad. Sci. U.S.A., 107:8531 (2010).
Chinnawirotpisan et al., Arch. Virol., 153:2225 (2008).
Deans et al., Cell, 130:363 (2007).
Deng et al., Acc. Chem. Res., 50:1059 (2017a).
Deng et al., Angew. Chem., 126:2421 (2014).
Deng et al., Chem. Sci., 1:3668 (2017b).
Dirks & Pierce, Proc. Natl. Acad. Sci. U.S.A., 101:15275 (2004).
Durbin & Whitehead, S. S. Dengue Vaccine Candidates in Development.
In Dengue Virus, Rothman. A. L., ed.; Springer: Berlin, Heidelberg, pg. 129 (2010).
Endo et al., Nat. Commun., 4:2393 (2013).
Endo et al., Sci. Rep., 6:21991 (2016).
Ferrell & Ha, Trends Biochem. Sci., 39:612 (2014).
Fowler et al., Nucl. Acids Res., 44:e48 (2016).
Gam et al., Nat. Commun., 9:2430 (2018).
Ghodasara & Voigt, Nucleic Acids Res., ------:------(2017).
Gibson et al., Nat. Methods, 6; 343 (2009).
Greber & Fussenegger, J. Biotechnol., 130:329 (2007).
Green et al., Cell, 159:925 (2014).
Green et al., Nature, 548:117 (2017).
Gubler et al., Am. J. Trop. Med. Hyg., 3.4:170 (1985).
Gubler, Trends Microbiol., 0.0:100 (2002).
Guo et al., Tumor Biol, 37:115 (2016).
Hadinegoro et al., Paediatr. Int. Child Health, 32:33 (2012).
Hayes et al., Trends Mol. Med., 20:460 (2014).
Haynes et al., ACS Synth. Biol, 1:99 (2012).
Hooley et al., Adv. Opt. Mater, 2:1109 (2015).
Hoynes-O'Connor & Moon, ACS Synth. Biol., 5:1441 (2016).
Huang et al., Sci. Rep., 6:22303 (2016).
Jang et al., Curr. Opin. Biotechnol., 33:1 (2018)
Jansson & Lund, Mol. Oncol., 6: 590 (2012).
Katzelnick et al., Science, 358:929 (2017).
Kim et al., ACS Synth Biol., 8: 601 (2019).
Kong et al., J. Virol. Methods, 138:123 (2006).
Lardo et al., Asian Pac. J. Trop. Dis., 2:134 (2016).
Lee & Moon. Methods, 143:58 (2018).
Lee et al., ACS Synth. Biol., 7:853 (2018).
Lee et al., Nucleic Acids Res., 4:2462 (2016).
Lin & Gregory, Nat. Rev. Cancer, 15:321 (2015).
Lisova et al., J. Mol. Recognit., 27:205 (2014).
Luo, Cold Spring Harbor Perspect. Biol., 9:a022137 (2017).
Messina et al., Nat. Neurosci, 19:835 (2016).
Miki et al., Cell Stem Cell, 16:699 (2015).
Moi et al., J. Gen. Virol., 25:591 (2014).
Moon et al., Nature, 491:249 (2012).
Munoz-Jordan et al., J. Clin. Microbiol., 47, 927 (2009).
Na et al., Nat. Biotechnol., 31:170 (2013).
Nana-Sinkam & Croce, Transl. Res., 157:216 (2011).
Ono et al., FEBS J., 278:1619 (2011).
Pardee et al., Cell, 159:940 (2014).
Pardee et al., Cell, 165, 1255 (2016).
Pardee et al., Cell, 167:248 (2016).
Pothoulakis et al., ACS Synth. Biol., 3:182 (2014).
Qi et al., Cell, 152:1173 (2013).
Rathakrishnan & Sekaran, Expert Opin. Med. Diagn., 1:99 (2013).
Reddy et al., Peer J., 5:e2970 (2017).
Ritchie et al., J. Phys. Chem. C, 111:175 (2007).
Sassen et al., Virchows Arch., 452:1 (2008).
Shopera et al., Nucleic Acids Res., 43:9086 (2015).
Soo et al., PLoS One, 11:e0154760 (2016).
Sriprapun et al., Asian Biomed., 9: 197 (2015).
Suzuki et al., Oncogene, 34:3085 (2015).
Suzuki, Int. J. Mol. Sci., 19:1901 (2018).
Tang et al., Anal. Chem., 90:10001 (2018).
Thum et al., Cardiovasc. Res., 2:562 (2008).
Urbich et al., Cardiovasc. Res., 79:581 (2008).
Wang et al., Synth. Biol., 3:------(2018).
Wang et al., ACS Synth Biol, 8: 1079 (2019).
Win et al., Proc. Natl. Acad. Sci. U.S.A, 104:14283 (2007).
Winkler et al., Nature, 419:952 (2002).
World Health Organization. Dengue and Severe Dengue Fact Sheet, World Health Organization, Geneva, Switzerland, 2012 (2016).
Wroblewska et al., Nat. Biotechnol, 33:839 (2015).
Wu et al., J. Clin. Microbiol., 39:2794 (2001).
Xie et al., Acta Biochim. Biophys. Sin., 50: 121 (2018).
Xie et al., Curr. Opin. Biotechnol., 40:139 2016.
Xiong et al., Nucleic Acids Res., 32:------ (2004) A
Yap et al., PLoS Negl. Trop. Dis., 5:e1046 (2011).
Yurke et al., Nature, 406:605 (2000).
Zadeh et al., J. Comput. Chem., 32:170 (2011).
Zuker, Nucleic Acids Res., 31:3406 (2003).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A gene therapy vector comprising a promoter operably linked to a nucleic acid sequence comprising an open reading frame encoding a prophylactic or therapeutic gene product and a 3′ untranslated region (3′ UTR), wherein the vector further comprises a first transcriptional regulatory region 3′ to the open reading frame that, when present in transcribed RNA, is capable of i) interacting with a short interfering RNA (siRNA) sequence which interaction inhibits translation of the transcribed RNA or ii) enhancing degradation of the transcribed RNA, thereby inhibiting the expression of the gene product, and/or wherein the vector further comprises a second transcriptional regulatory region 5′ to the open reading frame that, when present in transcribed RNA, is capable of interacting with a short activating RNA (saRNA) sequence which interaction allows for translation of the transcribed RNA, thereby allowing for expression of the gene product.

2. The vector of claim 1 wherein the vector has the first transcriptional regulatory region but not the second transcriptional regulatory region or wherein the vector has the first transcriptional regulatory region and the second transcriptional regulatory region.

3. The vector of claim 1 wherein the vector has the second transcriptional regulatory region but not the first transcriptional regulatory region.

4. (canceled)

5. The vector of claim 1 wherein the vector is a viral vector.

6. (canceled)

7. The vector of claim 1 wherein the transcribed RNA having the second transcriptional regulatory region forms at least one hairpin structure or wherein the interaction of the saRNA with the transcribed RNA having the second transcriptional regulatory region exposes the ribosome binding site or wherein the transcribed RNA having the second transcriptional regulatory region forms one to three or two to five different hairpins or wherein the transcribed RNA having the second transcriptional regulatory region forms at least one hairpin that overlaps the ribosome binding site and/or the first AUG in the transcribed RNA or wherein the second transcriptional regulatory region comprises a toe hold sequence, or any combination thereof.

8-11. (canceled)

12. The vector of claim 1 the first transcriptional regulatory region comprises more than one nucleotide sequence that when transcribed into RNA can bind more than one distinct siRNA or can bind a plurality of siRNAs.

13-14. (canceled)

15. The vector of claim 1 wherein the open reading frame encodes a therapeutic RNA, a therapeutic antibody, an anti-cancer gene product, a complement factor, an interleukin, a cytokine, or a hormone or wherein the gene product comprises an anti-EGFR antibody, anti-VEGF antibody, anti-VEGFR antibody, alpha 1-antitrypsin, catalase, superoxide dismutase, factor 9, IL-2R, adenosine deaminase (ADA), WAS, beta-globin, ABCD1, anti-CD19 antibody, anti-IgG antibody, anti-Siglec antibody, or FK506 binding protein.

16. (canceled)

17. The vector of claim 1 wherein the first transcriptional regulatory region comprises a sequence that binds to a siRNA having at least 80% nucleotide sequence identity with one of SEQ ID Nos. 4 to 6.

18-24. (canceled)

25. A method to control expression of a gene therapy vector in a mammal, comprising:
providing a mammal having the gene therapy vector of claim 1; and
administering to the mammal an amount of a composition comprising nucleic acid comprising sequences for the siRNA and/or the saRNA effective to alter expression of the gene product encoded by the vector.

26. The method of claim 25 wherein the open reading frame encodes a protein, a therapeutic RNA, a therapeutic antibody, a hormone, a cytokine, an interleukin, or a ribozyme.

27-28. (canceled)

29. The method of claim 25 wherein the composition comprises a DNA vector having sequences corresponding to the siRNA sequences or the saRNA sequences.

30-32. (canceled)

33. The method of claim 25 wherein the composition comprises liposomes nanoparticles or protein complexes comprising the nucleic acid.

34-35. (canceled)

36. The method of claim 25 wherein the composition is systemically administered.

37-38. (canceled)

39. The method of claim 25 wherein the composition is locally administered.

40. (canceled)

41. The method of claim 25 wherein the composition is a sustained release composition.

42. The method of claim 25 further comprising administering the gene therapy vector to the mammal.

43-46. (canceled)

47. The method of claim 42 wherein the vector is intravenously administered.

48. The method of claim 42 wherein the vector is locally administered.

49. The method of claim 25 wherein the mammal is a human.

50. A vector having homology arms flanking a second transcriptional regulatory region that when present in transcribed RNA is capable of interacting with a short activating RNA (saRNA) sequence, wherein the homology arms have sequences that correspond to gene therapy vector sequences that flank a site for insertion of the second transcriptional regulatory region or a vector having homology arms flanking a first transcriptional regulatory region that when present in transcribed RNA is capable of interacting with a short interfering RNA (siRNA) sequence, wherein the homology arms have sequences that correspond to gene therapy vector sequences that flank a site for insertion of the first transcriptional regulatory region.

51-53. (canceled)


SEQUENCE LISTING (21 sequences)

SEQ ID NO: 1 - DNA, 21 bp, Artificial Sequence
  A synthetic oligonucleotide sequence
aagatcgtcgtaagcgcgtaa

SEQ ID NO: 2 - DNA, 21 bp, Artificial Sequence
  A synthetic oligonucleotide sequence
aagttacgcgacttacgcgat

SEQ ID NO: 3 - DNA, 21 bp, Artificial Sequence
  A synthetic oligonucleotide sequence
acgatcgttcatatcgcgtat

SEQ ID NO: 4 - RNA, 21 nt, Artificial Sequence
  A synthetic oligonucleotide sequence
gaucgucguaagcgcguaauu

SEQ ID NO: 5 - RNA, 21 nt, Artificial Sequence
  A synthetic oligonucleotide sequence
guuacgcgacuuacgcgauuu

SEQ ID NO: 6 - RNA, 21 nt, Artificial Sequence
  A synthetic oligonucleotide sequence
gaucguucauaucgcguauuu

SEQ ID NO: 7 - DNA, 21 bp, Artificial Sequence
  A synthetic oligonucleotide sequence
ctattgacgtatgacgcgtaa

SEQ ID NO: 8 - DNA, 21 bp, Artificial Sequence
  A synthetic oligonucleotide sequence
caatccatcattacgcgttaa

SEQ ID NO: 9 - DNA, 21 bp, Artificial Sequence
  A synthetic oligonucleotide sequence
caatccatcaattacgcgtta

SEQ ID NO: 10 - DNA, 21 bp, Artificial Sequence
  A synthetic oligonucleotide sequence
aagatcccgattgtcgatcgt

SEQ ID NO: 11 - DNA, 21 bp, Artificial Sequence
  A synthetic oligonucleotide sequence
acgtcgtacgttaccgtacga

SEQ ID NO: 12 - DNA, 151 bp, Artificial Sequence
  A synthetic oligonucleotide sequence
ctggaggcttgctttgggctgtatgctgttacgcgtcatacgtcaataggttttggccactgactcgacttatgattatgacgcgtaacaggacacaaggccctttatcagcactcacatggaacaaatggccaccgtgggaggatgacaa

SEQ ID NO: 13 - DNA, 151 bp, Artificial Sequence
  A synthetic oligonucleotide sequence
ctggaggcttgctttgggctgtatgctgttacgcgtcatacgtcaataggttttggccactgactcgacttattatgatgacgcgtaacaggacacaaggccctttatcagcactcacatggaacaaatggccaccgtgggaggatgacaa

SEQ ID NO: 14 - DNA, 151 bp, Artificial Sequence
  A synthetic oligonucleotide sequence
ctggaggcttgctttgggctgtatgctgttaacgcgtaatgatggattggttttggccactgactcgactaattatattacgcgttaacaggacacaaggccctttatcagcactcacatggaacaaatggccaccgtgggaggatgacaa

SEQ ID NO: 15 - DNA, 151 bp, Artificial Sequence
  A synthetic oligonucleotide sequence
ctggaggcttgctttgggctgtatgctgttaacgcgtaatgatggattggttttggccactgactcgactaatcattttacgcgttaacaggacacaaggccctttatcagcactcacatggaacaaatggccaccgtgggaggatgacaa

SEQ ID NO: 16 - DNA, 151 bp, Artificial Sequence
  A synthetic oligonucleotide sequence
ctggaggcttgctttgggctgtatgctgtaacgcgtaattgatggattggttttggccactgactcgactaatcattattacgcgttacaggacacaaggccctttatcagcactcacatggaacaaatggccaccgtgggaggatgacaa

SEQ ID NO: 17 - DNA, 106 bp, Artificial Sequence
  A synthetic oligonucleotide sequence
aaaagatcgtcgtaagcgcgtaattttccatcaagaacaggccaccatggaaaattacgcgaacctggcggcagcgcaaaagatgggggtgcacgaatgtcctgcc

SEQ ID NO: 18 - DNA, 106 bp, Artificial Sequence
  A synthetic oligonucleotide sequence
aaaagatcgtcgtaagcgcgtaattttccatctagaagacgccaccatggaaaattacgcgaacctagccccagcccaaaagatgggggtgcacgaatgtcctgcc

SEQ ID NO: 19 - DNA, 106 bp, Artificial Sequence
  A synthetic oligonucleotide sequence
aaaagttacgcgacttacgcgatttttccatcaagaacaggccaccatggaaaaatcgcgtaacctggcggcagcgcaaaagatgggggtgcacgaatgtcctgcc

SEQ ID NO: 20 - DNA, 106 bp, Artificial Sequence
  A synthetic oligonucleotide sequence
aaaagttacgcgacttacgcgatttttccatcaagaagacgccaccatggaaaaatcgcgttacctagccccagcccaaaagatgggggtgcacgaatgtcctgcc

SEQ ID NO: 21 - DNA, 151 bp, Artificial Sequence
  A synthetic oligonucleotide sequence
ctggaggcttgctttgggctgtatgctgtaacgcgtaattgatggattggttttggccactgactcgactaattataattacgcgttacaggacacaaggccctttatcagcactcacatggaacaaatggccaccgtgggaggatgacaa